(19) United States
(12) Patent Application Publication
(10) Pub. No.: US 2005/0208475 A1
(43) Pub. Date: Sep. 22, 2005

(54) Process and apparatus comprising episcopic differential contrast (EDIC) microscopy plus epifluorescence microscopy (EF) for the detection or identification of biological materials on surfaces

(76) Inventors: Eric Best, Swindon (GB); Charles William Keevil, Salisbury (GB)

Correspondence Address: FROMMER LAWRENCE & HAUG, 745 FIFTH AVENUE- 10TH FL., NEW YORK, NY, 10151, US

(21) Appl. No.: 11/081,493

(22) Filed: Mar. 16, 2005

Related U.S. Application Data
(63) Continuation-in-part of application No. PCT/GB03/04004, filed on Sep. 16, 2003.

(30) Foreign Application Priority Data
Sep. 16, 2002 (GB) 0221467.4

(51) Int. Cl. C12Q 1/00; C12Q 1/70; C12Q 1/04

(52) U.S. Cl. 435/4; 435/5; 435/34

(57) ABSTRACT

The present invention relates to a microscopy system for rapid and sensitive detection and identification of biological material on surfaces by implementing episcopic differential contrast (EDIC) microscopy plus epifluorescence (EF) microscopy wherein the microscope incorporates a DIC prism in the nosepiece and long distance objectives so that the materials can be visualised without requirement for a coverslip or oil or water immersion. The system is particularly useful for the detection and identification of infectious diseases.

### CROSS REFERENCE TO RELATED APPLICATIONS

This application is a continuation-in-part of International Application No. PCT/GB2003/004004, filed on Sep. 16, 2003, published as WO 2004/025295 on Mar. 25, 2004, and claiming priority to GB Application Serial No. 0221467.4, filed Sep. 16, 2002.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a detection system and a microscopy method for the rapid and sensitive detection and/or identification of biological materials on surfaces, in particular the detection or identification of bio-hazardous materials and infectious diseases including amyloid plaque diseases. The invention also relates to a microscopy apparatus, reagents and diagnostic aids for use in the method.

### BACKGROUND OF THE INVENTION

The identification and treatment of highly transmissible disease such as Amyloid diseases including TSE (Transmissable Spongiform Encephalopathies) and Alzheimer's remain world-wide healthcare problems. With such highly transmissible diseases, the risk and concerns over contamination are major issues and there is a pressing need for the development of very stringent decontamination protocols. A limitation in the development of such protocols has been the lack of suitable methods for screening and detecting these highly transmissible disease agents on surgical instruments and other complex surfaces that are difficult to examine. There is presently no practical method used for the detection of possibly infectious prion material on instruments and work surfaces apart from crude visual inspection. There is also a need for improved diagnosis methods.

Transmissible spongiform encephalopathies (TSE), otherwise known as prion diseases, are fatal degenerative brain diseases involving conversion from the normal alpha helical PrP protein to the beta-pleated sheet PrP^{sc} isoform. Current epidemiological and research evidence suggests that the risk of PrP^{sc} transmission from TSE patients to other humans may be very low; nevertheless, TSE agents constitute a serious bio-medical hazard. A proven route of infection involves contaminated surgical instruments, particularly those in contact with neural tissues. Although government agencies, World Health Organization and other institutions have distributed some guidance for safe working and prevention of infection, these guidelines are not compulsory and are not harmonised globally. In the UK, guidelines recommending relatively strict standards for decontamination of medical instruments have been proposed by the Advisory Committee on Dangerous Pathogen, Spongiform Encepalopathy Advisory Committee, and the Department of Health.

Resistance of PrP^{sc} to sterilisation cleaning has raised concerns about decontaminating surgical instruments such as endoscopes that usually involves a preliminary treatment, rinsing, actual disinfection, final rinsing and storage. PrP^{sc} destruction requires prolonged autoclaving, which is only possible at the present time with the most recent rigid endoscopes. Indeed, there are general concerns about the efficacy of autoclaving, chemical disinfectants and the temperatures and contact times required. Many decontamination studies carried out thus far have involved solutions or suspensions of PrP^{sc} agent and may not reflect the behaviour of surface-bound infectivity. In one notable study, Zobeley et al (1999) used Scrapie mouse brain homogenate bound to stainless steel to show that PrP^{sc} was not removed after repeated washing with phosphate buffered saline; moreover, there was a 30-fold reduction in prion after treating with 10% formaldehyde solution for one hour. The prion contaminated steel still caused infection when introduced into normal mouse brain. In particular there is an urgent requirement for rapid, sensitive detection of general contamination of medical instruments and specific PrP^{sc} attachment, before and after cleaning.

### SUMMARY OF THE INVENTION

It has now been found that by using long distance objectives and incorporation of a DIC (differential interference contrast) prism in a microscope nosepiece, episcopic differential contrast (EDIC) and epifluorescence (EF) microscopy can be combined so that materials can be visualised on opaque surfaces without the use of coverslips and oil or water immersion.

Accordingly, the present invention provides a microscopy method for use in the detection or identification of biological material on surfaces by implementing episcopic differential contrast (EDIC) microscopy plus epifluorescence (EF) microscopy wherein the microscope incorporates a DIC prism in the nosepiece, infinity corrected optics and and long distance objectives so that the materials can be visualised without requirement for a coverslip or oil or water immersion.

Long distance objectives are high power objectives that have a working distance of greater than 1 or 2 millimeters.

Infinity Correction is composed of parallel optical paths, which enable the system to utilise a variety of different disciplines, to produce high resolution images within a range without the additional requirement for extra optics.

The surfaces which can be examined include curved, ridged, smooth opaque or semi -opaque, transparent, fibrous, rough or corroded surfaces. The method is particularly useful for examination of stainless steel instruments, surgical instruments, work surfaces, plastic surfaces, pipes or pipe biofilms, clothes, fabrics, food, grains, indwelling devices, biological samples, biopsy materials biofilms, membranes, interior of cells or exterior of cells. The ability to examine instruments including forceps, surgical knives or scalpels, rigid or flexible endoscopes, cytoscopes, applanation tonometer tips is a particularly useful development. In dwelling devises which can usefully be examined include contact lenses and catheters.

The invention enables the examination of a variety of biological material such as protein contamination and is particularly useful in the detection and identification of biohazardous materials, such as diseases including diseases caused by bacteria or viruses and amyloidogenic proteins. Disease materials able to be visualised according to the invention include helicobacter, campylobacteria, CMV, MRSA, TB, smallpox, and anthrax. The diseases can be diseases which affect humans or non-humans. Non human diseases include BSE, Scrapie or deer/elk Chronic Wasting Disease.

A useful aspect of the ability of the biological materials to bind fluorophores is that the viability/vitality of the disease materials can also be determined using suitable staining techniques.

Suitable staining techniques include CTC or DAPI and/or PI. This approach is particularly useful where the disease is cryptosporidium.

Other specific probes which can be used include monoclonal antibodies, peptides, nucleic acids or pseudonucleic acids. Fluorophore agents useful in the invention method are fluorescent thiazole derivatives, such as Thioflavine T or S. In one method general protein contamination is detected using Sypro Ruby fluorescent stain. The detection level on stainless steel is less than 1 picogram of protein.

In a further aspect, the present invention provides a microscope apparatus for use in the method. According to the invention, the microscope comprises an EDIC microscope with a high powered light system and a filter arranged in the light path so that light differences on the sample can be visualised. A suitable high powered mercury lighting system , is for example 100 watt. In a preferred microscope arrangement, an immunogold staining block in included.

The microscope of the invention can be readily adapted to provide a handheld or portable device or for use in a conveyor belt or adapted for use in a modified containment cabinet. Devices according to the invention are useful for screening in the water industry for the examination of biofilms, within medical establishments, contamination within the food industry, on food surfaces (e.g. salads), abbatoirs, veterinary practices, and dentistry practices. The invention also provides kits for use in diagnostic screening for prion disease in patients after tissue biopsy, or for quantitative assessment of the extent of contamination bound to surfaces comprising associated packs of reagents specifically designed to be used in conjunction with the method to enable visualisation of target material. The kits suitably comprise probes for the relevant biological materials and/or any necessary stains.

The invention further provides a system for the diagnosis of disease including prion disease or any other amyloidogenic disease within bodily fluid of the human or animal subject, blood, urine, cerebral-spinal fluid, non-neuronal tissues (including spleen, lymph node), in cells, including living cells. Other uses include a system for rapidly screening biofilms and assessing their contents.

Portable (handheld) or conveyor belt stage models can be used to enable the rapid scanning of large surface areas or numerous articles in very short periods of time. Another option is a quality control/safety scanner, able to rapidly visualise the structural integrity of opaque surfaces and tool/instruments there by quantifying the degree of pitting, scratching, etching or cracking that may have occurred. Other uses include a method of assessing or validation of the effects or effectiveness of cleaning or disinfection methods on surfaces.

Surfaces which can be examined according to the invention include curved, ridged, smooth opaque or semi -opaque, fibrous, rough or corroded surfaces. In the context of cells, surfaces can be interior or exterior. Cells include all types of cells including, animals and plants as well as microrganisms such as bacteria.

The present invention has applications in many environmental situations including environmental microbiology, entomology, biofilms, and in the investigation of disease. In particular, in relation to prion disease the present invention integrates the use of fluorescent marker techniques, monoclonal antibodies and advanced microscopy methods to produce a highly sensitive protocol for prion protein detection. This involves a generic rapid staining of general protein contamination and development of protocols for β-pleated sheet amyloid using fluorescent markers. This enables the identification of prion deposits on surgical instruments and work surfaces, as well as the possibility for its application on biopsy material. A useful aspect of the present invention is that it provides a pre-screen for use in selecting specimens for subsequent confirmation of the prion diagnosis using monoclonal antibodies.

The present inventors have shown that implementation of episcopic differential interference contrast (EDIC) microscopy, coupled with epi-fluorescence (EF) microscopy, is particularly suited to such contamination studies because there is no requirement for coverslips or oil immersion, and this method can rapidly visualize material on opaque and curved surfaces without the generation of artefacts associated with other techniques.

Initial studies on sections of brain and spleen have shown the fluorescent diagnostic procedures to be highly sensitive, with preliminary results indicating a detection level of less than 1 ρg of PrP^{sc} on stainless steel surfaces. With particular attention to Transmissable Spongiform Encephalopathies, there is now provided rapid, sensitive, visual and microscopic techniques for assessing general contamination and specific prion contamination to surgical devices. Such rapid visual and epimicroscopy techniques enable assessment of the contamination of materials having curved, ridged or smooth opaque surfaces such as surgical instruments, as well as assessing their presence in or on biofilms and cells as a method of detection and/or diagnosis. The facility for the detection of biohazardous contamination especially by Prion protein and other amyloidogenic proteins or other fluorophor binding contaminants on surgical instruments and work surfaces by the application of fluorescent reagents represents a major advance in technology.

Advantages of the present invention include those described below. The invention is useful for the screening of all surgical instruments for contamination in general, or more specifically for prion protein within medical establishments such as the UK National Health Service, Europe and world-wide. Other particular uses are for screening for contamination within the food processing industry, abattoirs, veterinary practices and dentistry practices. Also provided are quick diagnostic screening procedures for prion disease in patients after tissue biopsy.

One specific example involves four distinct approaches:
The immunohistochemical study of six monoclonal antibodies (SAF antibodies) raised against the PrP^{sc} protein. Their characterisation and suitability to detect PrP^{sc} on stainless steel.
The modification of a highly sensitive, fluorescent, protein stain, Sypro Ruby, to enable sensitive detection of general proteinaceous deposits on surgical stainless steel.
The modification and enhancement of specific β-sheet amyloid reagents (Thioflavine S and Thioflavine T) and their staining protocols to achieve sensitive detection of prion deposits on surgical stainless steel.
The visualisation of contamination on surgical instruments that have been processed through sterilisation and been deemed ‘clean’.

In particular the present studies demonstrate the ability of the microscopy technique and the reagents employed according to the invention for the rapid, sensitive, detection of prion contamination on surgical stainless steel to the sub-micron (<1 ρg protein) level.

A particularly useful aspect of the present invention is in relation to providing early screening methods of a number of diseases. For example enabling detection of infected cells or infective prion aggregates in the peripheral blood, lymphoid tissues or other bodily fluids of species infected with TSE diseases. Such screening methods can be used to determine the incidence of infection in the general population of these species and accordingly the stage of disease progression in infected individuals. Other diseases which can be monitored in this way include CMV.

There is presently a lack of tests suitable to use to detect infected sheep, cattle and humans, from readily accessible samples, at an early stage in the progression of the disease. Indeed only tests to be used at post-mortem have been approved by the European Commission (EC). There is, therefore a great need to design diagnostic tests which can be used to identify infected individuals long before the terminal clinical and veterinary symptoms become apparent. Such a test enables controls to be put in place to avoid cross-contamination between individuals in the same species and between species.

The present studies demonstrate sub-micron amyloid plaques on stainless steel, corresponding to sub-picogram levels of contamination. This three-dimensional capability means that the technique is very good for z-direction detection and has been shown to be able to examine level differences of <10 Å; thus enabling detection of high extinction factor values, increasing image contrast and brightness. Devices according to the invention can also be used in the water industry for the examination of biofilms in addition to the screening of surgical instruments for contamination in general or more specifically for pathogens such as MRSA, TB, or prion protein within medical establishments or elsewhere and screening for contamination within the food industry, abattoirs, veterinary practices, dentistry practices.

The apparatus and method also allows for the production of a quick diagnostic screening test for prion disease inpatients after tissue biopsy. Further uses include clinical pathology or any field requiring analysis for unwanted microbial contamination including pathogens or biohazardous materials. Campylobacter are readily visualised using this system. Also in the field of food microbiology the system has many uses eg, spot sampling for microbial contamination of eg salads). The system has uses in the area of plant pathology, for example the in-field diagnosis of plant diseases, especially those caused by various strains of micorganisms. The invention also offers uses in the area of forensics where rapid on site identification of deposits by non-destructive methods is important. Similarly the rapid identification of biohazardous materials for securing personal and public safety is particularly valuable.

Microscopy

Existing microscopes are unable to provide the necessary images as required by the present invention. For example Laser confocal microscopes are expensive, slow, require cover slips or oil or water immersion and are unable to visualise curved surfaces. Also the electron microscope is very expensive, very slow, has high running costs, desiccation is required and applications and uses are limited by the size of the object of interest.

Conventional DIC microscopy implements transmitted light and specimens on glass slides with coverslips and oil immersion lenses. This is clearly inadequate for contamination detection on opaque, curved instruments or work surfaces.

With this in mind the present invention provides an improved microscope that implements Episcopic DIC illumination, hence EDIC, to visualise the sample thus providing many advantages over traditional techniques. The long distance lenses used are normally only applied in metallurgical applications and hitherto not biological applications. The present invention enables the apparatus to perform rapid, sensitive, non-contact screening of opaque and/or curved surfaces without the application of coverslips or immersion; thus making it ideal for inspection of clinical instruments. The apparatus can be adapted and adjusted to allow task specific operations. For example the rearrangement or removal of the stage allows the production of a handheld or portable device or a device suitable for isolation, for example in a containment cabinet.

A microscope for EDIC microscopy has been described in Keevil, C. W. and Walker, J. T Normarski DIC microscopy and image analysis of biofilms’. Binary; 4:93-95,1992 and Roger, J., Keevil, C. W. Immunogold and fluoroscein immunolablelling of Legionella pnemohila within an aquatic biofilm visualised by using episcopic differential interference contrast microscopy’. Appl. Environ. Microbiol.,:58; 2326-2330, 1992.

According to the present invention, an improved episcopic DIC microscope has been constructed, as described in more detail below, which has successfully visualised low level contamination by infected brain homogenates and brain slices of stainless steel surfaces without the need for coverslips or oil immersion and enable the visualisation of surface contamination on curved, ridged or smooth opaque, or semi-opaque or transparent surfaces.

The integration of long distance objectives and an ability to work with fluorescent as well as bright field media and its non-requirement for coverslips or oil or water immersion enables the system to produce hitherto unprocurable images of surface materials and contaminants.

The microscope technique implements episcopic differential contrast (EDIC) methodology in addition to episcopic fluorescent (EF) microscopy. The system adapts conventional Nomarski microscopy to incorporate episcopic imaging by redesign of the light path and microscope components.

EDIC implements the destructive/constructive nature of light waves. The source light is split into two polarised parallel beams before it reached the specimen. Having transversed the object the wave paths of the two beams have been altered in accordance with the specimen's thickness, slopes and refractive index. This variation causes interference between the light beams and allows detail to be visualised in a pseudo three-dimensional appearance. This enables the operator to not only visualise the object of interest but also gives an indication of the position of any such object.

For example, one new microscope (with reference to FIGS. 1, 2 or FIGS. 40, 41) includes a number of additional individual improvement features
DIC prism above stage, rather than individuals below
Improved EDIC/EF cube slider for image superimposition
Improved non-contact, long distance lenses
Better ergonomic configuration of polarizer, prism, zoom and stage
Fully automated stage for x, y and z scanning
Automated software to focus curved images
CCD camera imaging optics for improved 2D and 3D presentation
Facility for confocal adaptation without laser requirement.

Low power objectives (<×20) have the inherent ability to work at relatively long distances from samples. This is not true for most high power lens (>×20) and special long distance objectives, that have a working distance of greater than one or two millimetres, have been applied to the system.

By comparison with the earlier 1992 EDIC system referenced above, the present invention microscope optical configuration (see FIG. 1) is based upon infinity corrected optical system. The present system enables longer working distances with objective lenses to be achieved, a significant improvement from the mercury light source due to the infinity corrected light path and optical system, i.e. enabling better illumination.

With the present invention the device for EDIC is correctly positioned in the nosepiece just above the objective lenses enabling quality EDIC to be achieved without the use of extra prisms for this technique to take place and extra optical glass wear to magnify the image to the next optical requirement, in this case epi fluorescence.

This infinity corrected EDIC system also enables you to use this on the current infinity corrected lenses of choice whether they are DIC specified or not which again is a improved technique enabling the user to use lenses that are specified DIC this means slightly better quality or those which are not specified the new improved EDIC enables you to work using any biological or material science objective.

The separate interaction with the new infinity corrected system whether using immuno-gold staining block in conjunction with EDIC or being used separately enables quality images to be achieved which compliment each other by providing 100 per cent more illumination when both are engaged in the epi illumination format with excellent spectral colours being achieved through the entire spectrum.

The present system can use new improved epi fluorescence filter combinations with 25 mm filters and new improved filter combinations cubes i.e. new coatings. (e.g. Chroma filters, USA) enabling a wider saturation of required excitation of the particular wavelength and band pass required.

Unlike the present system, the earlier 1992 microscope was of a 160 mm tube length optical configuration, with an extra 50 mm insert required making 210 mm for all episcopic material science lenses. The biological ones were 160 mm. So when using material sciences lenses of long working distance these were the correct magnifications, when using biological these were giving more magnification due to the 50 mm insert used to obtain the correct optical path for material science lenses and the epi fluorescence system. The new infinity corrected system results in a superior match of optical configurations.

In 1992 the EDIC system was based on a rotating analyser which worked in conjunction with the immuno-gold staining block. This analyser did not contain the Nomarski principal as now described in the new improved model. Instead it obtained the Nomarski interference imaging by the use of a prism directly behind the objective lens which could be engaged by a lever placing it in or out of the optical path.

The material science long working distance objective had its own individual prism, the biological lenses had no prisms hence only used the IGS block and analyser to obtain polarisation and epi fluorescence imaging so limiting the 1992 EDIC system to the lenses that had the Nomarski prism in situ. In contrast, the present system allows any chosen objective to be used.

In the 1992 EDIC, the Nomarski interference imaging was obtained by the use of a prism directly behind the objective lens which could be engaged by a lever placing it in or out of the optical path. The rotating analyser also sat in the position above the fluorescent filter blocks and was rotated to obtain the Nomarski principal. This configuration of EDIC gave reasonable results but was not specified by the manufacturer as a current working system. However the configuration according to the present invention using infinity corrected optics is capable of far superior optical quality.

The original filters integrated into the filter blocks were only 18 mm limiting their epi fluorescence function whereas it is now possible to use new filters integrated into new filter blocks which are 25 mm. The extra available lighting in the new infinity corrected system coupled with the larger filter area provides excitation and illumination of specimens far beyond that possible with the 1992 EDIC.

The 1992 EDIC included four filter blocks which were engaged into the optical pathway via a push pull rod, however the problem with this was that engaging the filter block correctly into the optical path could sometimes be a problem especially to a new user. This has been overcome and more versatility added by designing the new system so that four blocks easily click into place. The new system can also readily accommodate further e.g. six filter blocks if required, also infinity corrected.

As can be seen, the new system provides superior optical quality and versatility, in particular through the use of infinity corrected optics, fluorescent coatings, and configuration changes that compliment EDIC without extra glass wear and Nomarski prisms which limited the use of the earlier 160/210 mm 1992 EDIC configuration.

### BRIEF DESCRIPTION OF THE DRAWINGS

Various preferred features and embodiments of the present invention will now be described in more detail by way of non-limiting example and with reference to the accompanying Figures, in which:

FIG. 1 shows a schematic of an EDIC microscope.

FIG. 2 shows an EDIC microscope.

FIG. 3 shows a murine section indicating PrP^{sc} positive regions for ME7.

FIG. 4 shows PrP epitopes recognised by the SAF Mab antibodies in relation to the protein.

FIG. 5 shows Sypro Ruby excitation/emission spectra.

FIG. 6 shows thiazole derivative structures.

FIG. 7 shows the versatile nature of the EDIC microscope.

FIGS. 8a-8c show activated microglia on stainless steel brain sections.

FIGS. 9a-9c show positive SAF areas in dentate gyrus, comparable to 6H4.

FIGS. 10a-10c show a positive SAF signal in the CA3 region of the hippocampus, similar to 6H4.

FIG. 11 shows a low magnification EDIC image of brain contamination on stainless steel.

FIG. 12 shows a high magnification EDIC image of brain contamination on stainless steel.

FIGS. 13a-13c show Sypro Ruby staining of brain contamination on stainless steel, showing proteinaceous material.

FIGS. 14a-14c show low magnification EDIC/EF images showing detection of protein deposits remaining after washing and stained with Sypro Ruby.

FIGS. 15a-15c show high magnification EDIC/EF images showing detection of protein deposits remaining after washing and staining with Sypro Ruby.

FIGS. 16a-16c show Thioflavine-positive regions in 10 μm sections of infected dentate gyrus on stainless steel.

FIGS. 17a-17c show Thioflavine-positive area in 10 μm sections of infected CA3 region of hippocampus on stainless steel.

FIGS. 18a-18c show Thioflavine-positive regions in 10 μm sections of infected thalamuss, comparable to 6H4 Mab staining on stainless steel, compares directly the immunohistological staining by 6H4 Mab (18a) and the Thioflavines (18b, 18c) within the thalamus of prion positive sections placed onto surgical stainless steel tokens.

FIG. 19 shows Spencer Wells Forceps.

FIGS. 20a-20c show contamination found on outside of the Spencer Wells forceps.

FIG. 21 shows Zoellner Sucker with close up indicating exposed lumen.

FIGS. 22a-22c show Spyro Ruby staining of contamination on lumenal surface.

FIG. 23 shows a cystoscope set.

FIG. 24 shows contamination at the tip of a 70° cystoscope.

FIG. 25 shows contamnination in the middle of 70° cystoscope.

FIG. 26 shows contamination on the lumenal surface of cystoscope set obturator sheath.

FIG. 27 shows contamination on the outside of cystoscope set obturator sheath.

FIG. 28 shows contamination at the tip of cystoscope obturator.

FIG. 29 shows brain sections.

FIG. 30 shows peripheral blood mononuclear cells stained with monoclonal antibody against the early Pk65 protein of Cytomegalovirus (CMV), Microgen Bioproducts CMV Antigenaemia kit (M83).

FIG. 31 shows peripheral blood mononuclear cells stained with monoclonal antibody against the early Pk65 protein of Cytomegalovirus (CMV), Microgen Bioproducts CMV Antigenaemia kit (M83). Configuration 1. (See Table 3.)

FIG. 32 shows a high power fluorescent image of peripheral blood mononuclear cells stained with monoclonal antibody against the early Pk65 protein of Cytomegalovirus (CMV), Microgen Bioproducts CMV Antigenaemia kit (M83). Hoffman configuration. (See Table 3.)

FIG. 33 shows Thioflavin T-stained Dentate gyrus showing prion amyloid aggregates (FITC filter block) at 1000× magnification. Configuration 1. (See Table 3.)

FIG. 34 shows a composite of Thioflavin/spyro ruby, spyro ruby stained (TRITC filter block). Configuration 1. (See Table 3.)

FIG. 35 shows droplets excreted by fruit fly egg after laying (EDIC microscopy) at 1000× magnification. Configuration 3. (See Table 3.)

FIG. 36 shows a single focal plane of drinking water biofilm on polyethylene pipe at room temperature using EDIC/Confocal microscopy (Optigrid). Configuration 6. (See Table 3.)

FIG. 37 shows a composition of ten stacked images of drinking water biofilm on polyethylene pipe at room temperature using EDIC Confocal microscopy (Optigrid). Configuration 6. (See Table 3.)

FIG. 38 shows 400× magnification of a spleen in bright field. Configuration 8. (See Table 3.)

FIG. 39 shows 100× and 1000× magnification of a spleen in Hoffman modulation contrast. Configuration 9. (See Table 3.)

FIGS. 40 and 41 show photographs of the microscope.

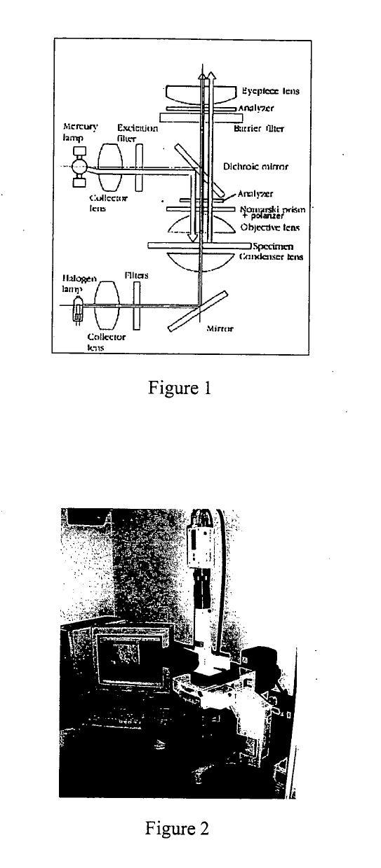

### DETAILED DESCRIPTION

The configuration of the microscope of the present invention is based upon a mixture of biological and material science requirements with an emphasis on looking at contamination on opaque specimens. This microscope has the ability to mix fluorescence and DIC (Differential interference contract) in the incident mode (light from the lamphouse on the top of the microscope through the optical path down to the objective onto the sample then back up to the eyepieces to receive the image of choice).

This has been achieved by using a high powered lamp with a light intensity power of 270CD (candela) such as a mercury lamp, suitably a 100 Watt mercury lamp. Using this system it is possible to supply light to the fluorescence filter cubes in conjunction with using the same light to drive the DIC image contrast prism system. This is possible through the use of a customised polarised filter cube that is positioned into the light path (for example immuno-gold staining filter block), which then enables the user to establish light difference on the specimen that allows detail to be visualised in a pseudo three dimensional appearance. Further technical information is as follows.

DIC microscopy implements the destructive/constructive nature of light waves from a mercury light source the light is split into two polarised parallel beams before it reaches the specimen, having transferred the object the wave paths of the two beams have been altered in accordance with the specimen's thickness slopes and refractive index. This variation causes interference patterns between the light beams and allows relief and light difference to be achieved, hence the user can see peaks and pits giving a 3D appearance. This three dimensional capability allows the excellent Z direction detection and has been shown to be able to examine level differences of 10 angstroms this enables detection of high extinction factor values, increasing contrast and depth of field in the specimen.

This microscope overcomes the problem of placing specimens onto glass slides with cover slips and oil immersion lenses which for contamination detection on opaque curved instruments or work surfaces, transmitted light running through the specimen from below will not resolve surface contamination. Accordingly, episcopic DIC illumination with mercury lighting system e.g. EDIC to visualise the sample gives many advantages over traditional techniques.

System Features

Particular features which each provide significant advantages over existing systems are described below.

A DIC prism incorporated into the nosepiece: Two phase contrast position control enables the fine tuning of surface contamination and relief.

Improved EDIC/EF cube slider: Image superimposed blending two episcopic images for greater contrast and relief, coupled with the newly researched fluorochromes that also are used in this system. This epi fluorescence package provides a very powerful research and analytical system.

The use of EDIC is a two-part process. Firstly, the use of an immuno-gold staining block IGS, which is a polarizer and cross analyser, gives direct polarisation to the sample, hence providing diagnostic biorefringence to any structure that its light path can be changed once it engages the specimen. this is a stand alone technique, epi polarization via a mercury HBO lamphouse. This IGS block is also required when engaging the DIC prism as the requirement for the EDIC is parallel light. The IGS block provides this, hence the two work together. Improved non-contact, long working distance lenses mixing metallurgical with biological objective lenses by way of an integrated nosepiece is an improvement over an uncoordinated lens nosepiece which is unable to acquire the image of choice because of the movement that needs to take place between the selection of the required lens via the nosepiece.

Configuration of Better ergonomic configuration of polarizer prism and a new zoom system according to the invention (the “Best-Keevil” zoom system) that can reduce or increase the overall magnification from a factor of 0.8× through to 2× so enabling a 40× lens in the eyepieces 400×, this having an eyepiece range of magnification from 320× to 800× using just one objective lens for both biological and material science is a useful addition in obtaining the correct image requirements whilst using a scanning stage.

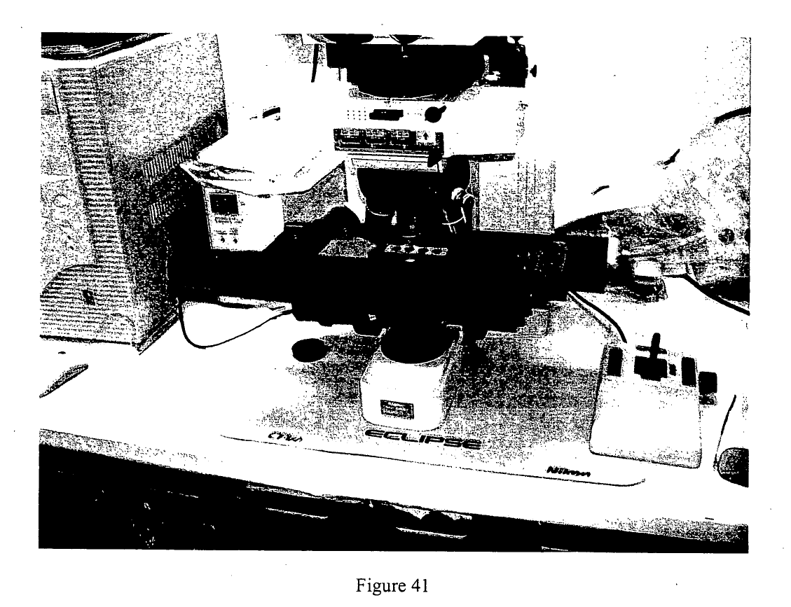

An 8 slide stage with glass insert for reflective light microscopy is provided. A preferred design features the drive motor in the Y position moved to the opposite corners allowing for greater freedom of movement for the operator. When a microscope needs to be isolated then the position of the stage is a problem due to protruding parts. A further advantage of the new configuration is that it deals with the protrusion of this part which then enables the microscope to be isolated, for example when placing the microscope in a ACDP (Advisory Committee on Dangerous Pathogens) category 1,2 or 3 containment cabinet. Other features include moving the control electronics input of this stage via computer from the right hand side to the left hand side. See FIG. 40 and 41

The system uses fully automated computer scanning stage in XYZ axes or direction including software to focus on curved images, also dedicated software to establish a cell bar code reader, for counting cell's per square mm for analysis and research, and further software to perform two spatially identical scans that are designed to map surface properties under fluorescence and DIC. The images will be merged/overlaid with its composite partner image.

Vision System

Cool snap charge coupled device (CCD) low light camera system for improved 2D and 3D preparation, the interfacing optics from the microscope trinocular head to the CCD camera have been designed according to the invention to embellish the lighting and images contrast quality produced by the optics and imaging system thus maximizing surface quality.

The present invention also relates to the introduction of a confocal adaptation without laser requirements. The introduction of the Optigrid real time digital optical sections for 3D mapping of textured specimens [Optigrid technology is described in GB 2,338,858 and U.S. Pat. No. 6,376,818] as shown herein clearly provides increased and excellent resolution.

Also developed in conjunction with IGS (immuno-gold staining) and EDIC is the customised (diascopic differential interference contrast (DDIC) diascopic or transmitted light from below the specimen for contamination that required fluorescence and EDIC from light looking down onto the specimen to DDIC looking up through the specimen, the contrast technique Hoffman contrast is used for this. (U.S. Pat. No. 4,200,353 Modulation contrast microscope). Using both techniques at the same time allows enhancement of the 3D relief of the EDIC coupled with the Hoffman 3D relief in the DDIC.

The IGS as a stand alone polarizing system incorporated into the EDIC in the episcopic format coupled with the Hoffman polarized technique in the diascopic format adds considerable resolution and contrast to a translucent specimen. These can be used in combination or separately. By adding the Thales-Optem optigrid real time digital optical sectioning as referred to above enables resolution beyond the capability of the microscope and the lenses. Due to the combined techniques listed above the confocal is able to establish resolutions on samples now that a stack of images can be combined to give one sharp image, whilst taking light from diascopic and transmitted light simultaneously.

Hoffman

The lens used for the Hoffman with its condenser is polarised to provided only the grey part of the spectrum whereby the light from the EDIC/fluorescence is providing the full spectrum of colours, hence the user can balance this system to work in such a way as to maximised the image texture on the specimen to define the contamination levels by altering the variation between the two relief techniques.

‘Modified’ Hoffman

The Hoffman 40× lens is able to see high contrast and resolution on transparent, unstained and living cells, as this is the only Hoffman lens on this microscope system when we move to another lens but without moving the polarized facility on the sub stage condenser we are still able to achieve with good resolution a “pseudo Hoffman” image on biological and material science lenses in transmitted light format that do not have the Hoffman facility. In essence this provides excellent relief on the specimen in transmitted light enabling height differential to be established picking out peaks and pits.

In summary the above features and adaptations provide a microscope to perform rapid sensitive, non contact screening of the opaque and curved surfaces without the application of the cover slips or oil. So making the microscope ideal for the inspection and analysis of opaque substrate and clinical surgical instruments. The microscope according to the invention represents a new revolutionary way in which contamination of surface structures Bio films etc. is viewed.

The further versatility of the apparatus can be illustrated with reference to Table 3 which provides an overview of the different configurations in which the microscope can be set up.

Episcopic (epi) Illumination

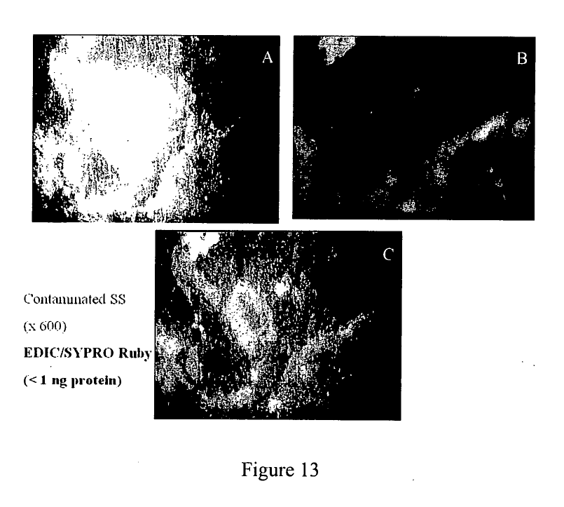

Under episcopic illumination configurations, the system has the ability to view any type of surface, either in ‘white’ light and under fluorescent excitation without the requirements for any major adjustments to the microscope set up. This allows both epi fluorescent (configuration 1) images and EDIC ‘whitelight’ (config. 3) or epi-polarised (config. 2) to be captured of the same area on interest. An example of the power of this is clearly shown in FIG. 13 where protein contamination of stainless steel surface is displayed under both fluorescence (FIG. 13a) and EDIC (FIG. 13b) illumination. The subsequent application of the advanced software enables a composite image of the two configurations to be displayed and as such the accurate differentiation of contaminants to be achieved (FIG. 13c).

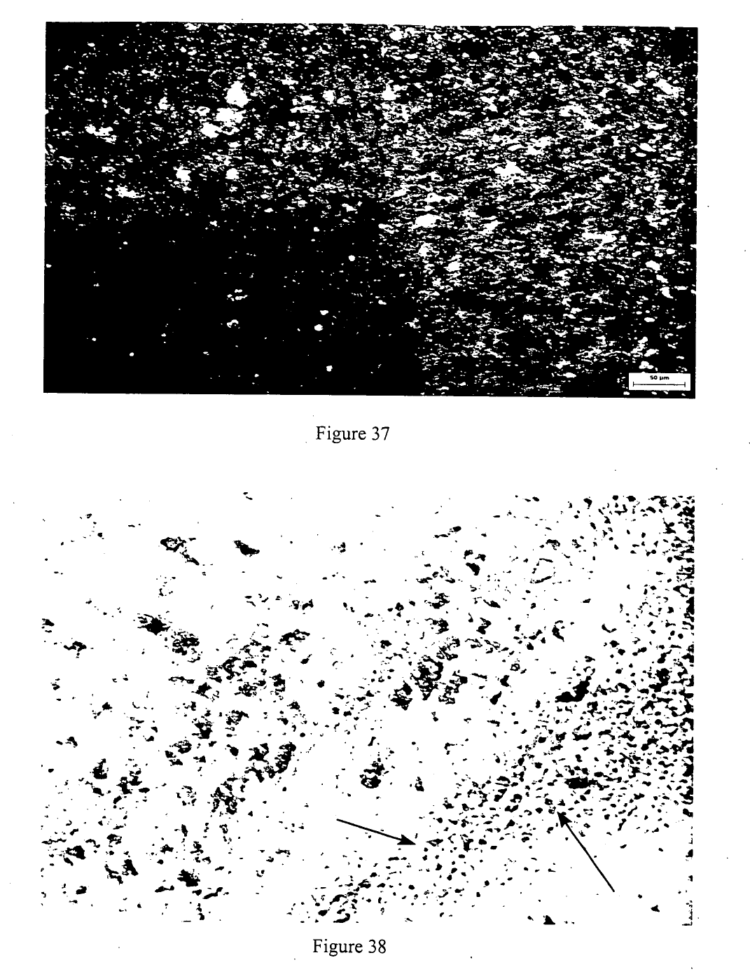

The addition of the ‘Thales-Optem optigrid’ enables an extended field of depth and allows the system to scan multiple planes of the object in a similar way to laser confocal but without the requirement for expensive additional equipment. This system enables the visualisation of biofilms on polyethylene pipe sections either using ‘whitelight’ (config. 6/7, FIG. 37) or fluorescence (config 6).

Transmitted Illumination

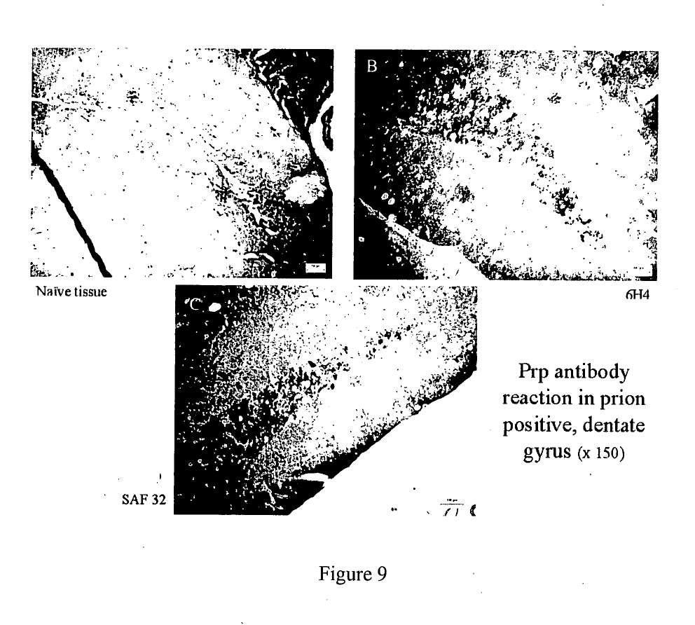

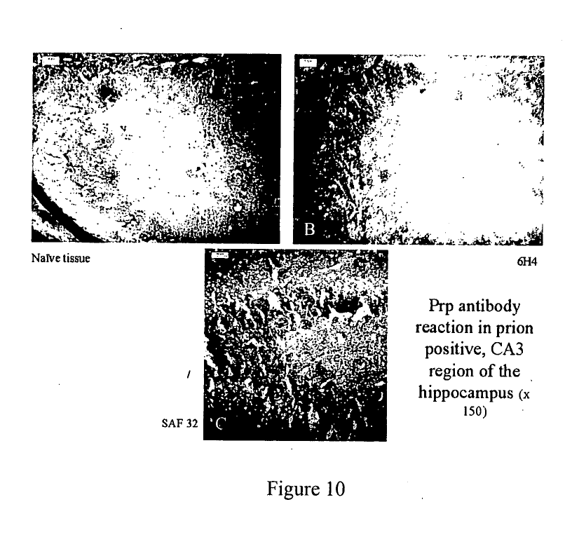

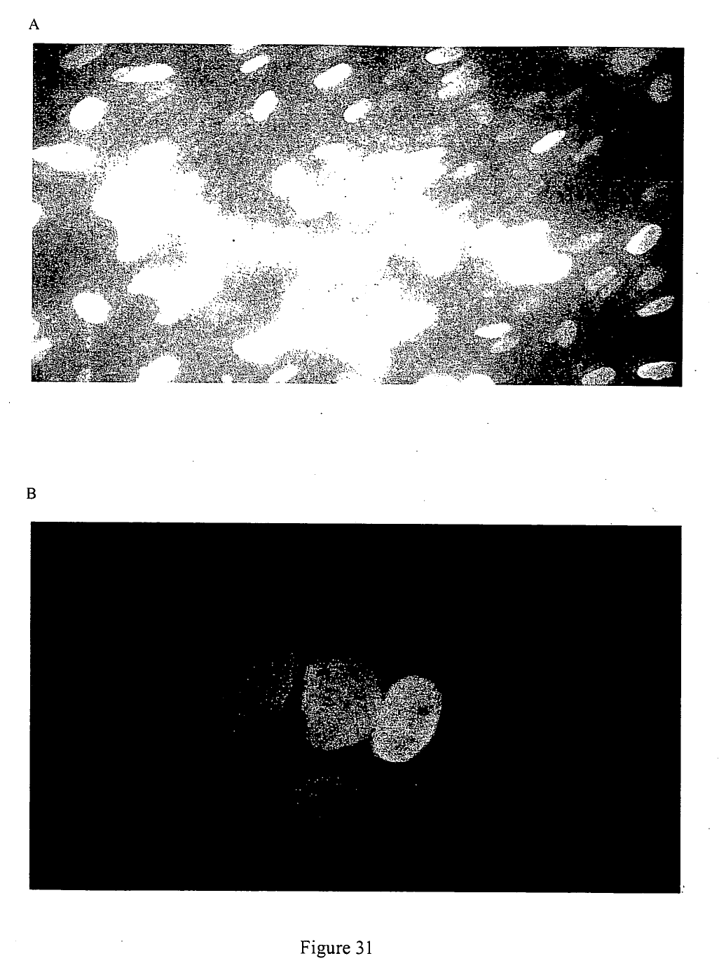

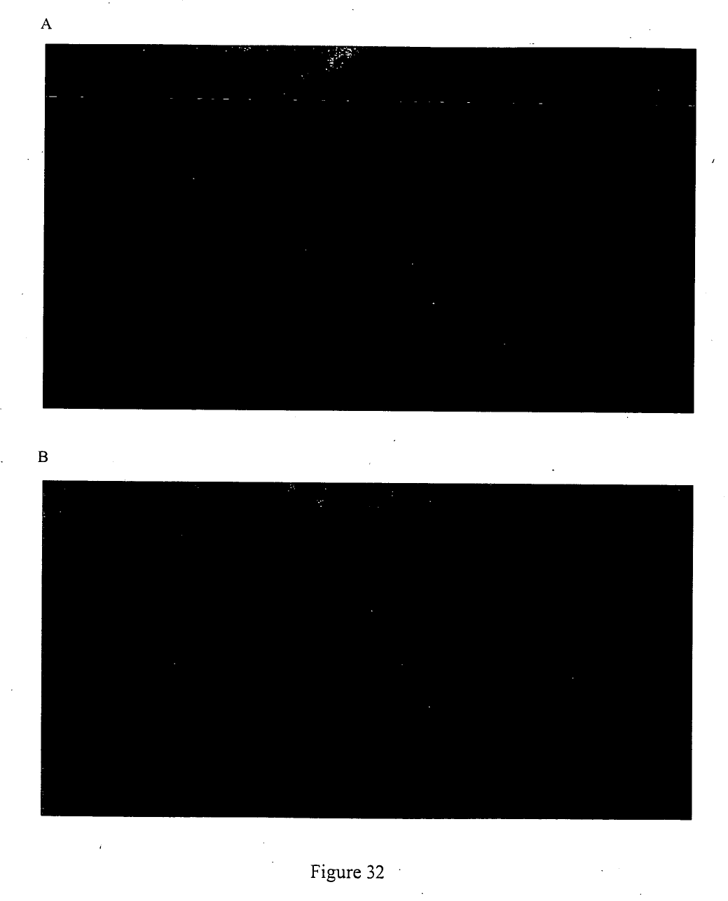

Transmitted illumination can only visualise samples in or on translucent media. Clearly the brightfield configuration (config 8) enables traditional microscopy to be performed such as traditional immunohistochemical staining (FIGS. 9 and 10). Hoffman modulation (config 9) enables relief to be visualised, it does not suffer the artefacts produced by plastic in other illumination methods and can be applied to such areas as the visualisation of cells (FIG. 31)

Transmitted Light and Epi-Illumination

The ability of the system to combine light sources allows the flexibility to adjust the system in order to produce the best quality images for the media and subject involved in both fluorescent and whitelight modes. An example of this is the ability to visualise bacteria such as Campylobacter jejuni (config 13). Campylobacter is a bacterial pathogen that is the most important cause of gastroenteritis worldwide and transmitted through faecal contamination.

The invention will now be described by way of further examples which are meant to assist one of ordinary skill in the art in carrying out the invention and are not intended in any way to limit the scope of the invention.

### EXAMPLES

Microscope

The system is based around a Nikon Eclipse ME600, modified as required and fitted with a combination of fluorescent, metallic and Hoffman modulation contrast objectives as described below:

| | |
|---|---|
| 10X biological objective (Nikon) | Plan x10, NA 0.25, WD 10.5 |
| 40X fluorescent objective (Nikon) | Plan Fluor x40, NA 0.6, WD 3.7-2.7 |
| 40X HMC (Modulation optics Inc) | HMC x40, NA 0.55 WD 1.7-2.7 |
| 50X metallic objective (Nikon) | LU Plan x50, NA 0.55, WD 10.1 |
| 100X metallic objective (Nikon) | L Plan x100, NA 0.7, WD 6.5 |

The system is able to house 4 filter blocks, one immuno-gold staining block for white light EDIC/epi-polarised illumination and 3 fluorescent filter blocks from Nikon and had the codes: UV-2E/C, BV-2A, and G-2E/C.

General Contamination and Cleaning

Surfaces were contaminated by passing fresh-frozen, un-embedded naive brain tissue block across the tokens surface. Direct assessment after contamination by brain material was undertaken by EDIC microscopy. Protein was detected using Sypro Ruby (SR) assessment (Molecular Probes, Oreg., USA). The samples were fixed in aqueous 7% acetic acid, 10% methanol for 15 minutes and then washed with phosphate buffered saline; three times, five minutes each wash. Samples were incubated with SR for 10 minutes, and then washed in filtered de-ionised water for 10 minutes.

For instruments no fixation (aqueous 7% acetic acid, 10% methanol for 15 minutes) was applied.

For the cleaning step, the samples were washed in water for 5 minutes at room temperature, they were then physically cleaned by rubbing the token surface with a surgical swab in the presence LabGuard scrub (Day-Implex Ltd, Essex, UK) for 2 minutes, and then washed in three changes of water for 5 minutes each. The tokens were then stained with Sypro Ruby and examined under the microscope.

Immunocytochemistry

Formal saline-fixed tissue was used for immunocytochemistry. For PrP^{sc} detection the sections were pre-treated to destroy PrP^{c}. This consisted of 15 minutes hydrated autoclaving, followed by 5 minutes with formic acid (>95%). The protocol then used the mouse on mouse kit²⁹ (Vector labs). The primary antibody 6H4 (Prionics), a monoclonal raised against the C-terminus region of the PrP protein, was left to incubate overnight at 4° C. with a concentration of 1:4000. Positive staining was visualised using a diaminobenzidine (DAB) as the chromagen and counterstained with hematoxylin.

Thiazole Staining

Thioflavine S (Sigma)—Sections of fresh tissue on glass slides or steel coupons were fixed in 4% paraformaldehyde (w/v) for 10 mins at 4° C. After washing with PBS the sections were incubated with Thioflavine S (0.01% w/v solution) for 10 minutes at room temperature. Subsequently the slides were washed in decreasing alcohol concentrations and the section covered with an aqueous fluorescent mounting medium (DakoCytomation).

Thioflavine T (Sigma)—Sections of fresh tissue on glass slides or steel coupons were fixed in 100% ethanol at 4° C. for 10 mins. After washing with PBS the sections were then incubated with Thioflavine T (0.03 % w/v solution) for 10 mins at room temperature. The samples were then washed with 1% acetic acid in de-ionised water for 40 minutes and the section covered with an aqueous fluorescent mounting medium (DakoCytomation).

### Example 1

### Prion Detection

1.1 Prion Propagation

Female C57Bl/6J mice were kept in-groups of five or six in plastic cages in a temperature-controlled room (21° C.) with a twelve-hour day/night cycle. They had free access to water and food.

Positive animals were those injected with 1 microlitre of 10% (w/v) ME7 brain homogenate, stereotaxically into the right dorsal hippocampal region of the brain (co-ordinates from bregma (the point on the top of the skull at which coronal and sagittal sutures meet): anterior-posterior—1.94 mm, lateral—1.5 mm, depth—1.5 mm).

Two sets of control animals were implemented:
Naïve, C57BL/6J mice left without injection and
Normal Brain Homogenate (NBH) which were those mice injected stereotaxically into the right dorsal hippocampus with 10% (w/v) brain homogenate derived from normal C57BL/6J mice.

All animals were then sacrificed at 19-21 weeks post inoculation (or equivalent time period for the naive group) and the tissue was either fixed with formalin by perfusion, or fresh frozen on liquid N₂, dependant on the testing method to be implemented.

1.2 Histochemistry

Primary investigations were conducted on thin tissue sections, thus establishing:
Defined areas of the mouse brain with known pathology, microglia activation and prion accumulation.
Known prion amyloid plaque histochemistry
Established sectional parameters: 10 μm sections (which dry to approx. 5 μm thick), equating to less than 1 cell thick
The ability for placement on stainless steel to calibrate EDIC/EF
   and therefore allowing the validation of the microscopy principle.

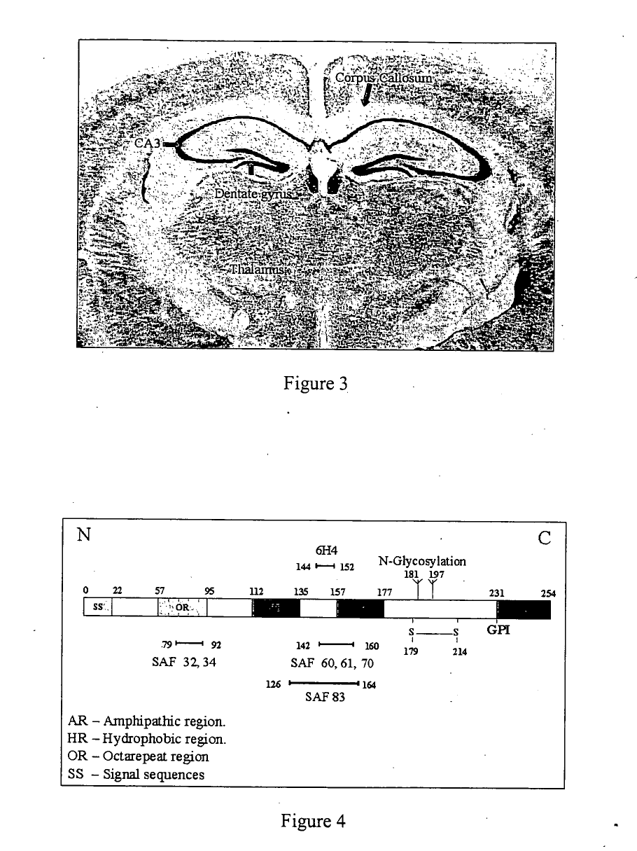

The ME7 model has well defined PrP^{sc} characteristics^{[8-15]} with large concentrations of the abnormal protein forming within sections of the hippocampal region of the brain. In particular, dense areas of PrP^{sc} deposition occur within the hilus of the dentate gyrus and following the mossy fibres of the CA3 region of the hippocampus. Additionally there are distinctive dense circular amyloid plaques found within the thalamus and occasionally above the corpus callosum (FIG. 3).

1.3 Protein Quantification of Neural Tissue

The protein content of the neural sections was determined (Appendix 1) both by i) theoretical derivation of published data of mouse brain and ii) protein assay of individual sections using the Bio-Rad total protein assay method. Close agreement between the methods was observed.

FIG. 3 Shows a Murine Section Indicating PrP^{sc} Positive Regions for ME7.

1.4 Antibody Investigations

1.5 Microglia

The activation of microglia within prion infection has been well documented;^{[16-25]} indeed, the up-regulation of such monocyte derived bodies is such that their involvement in the apoptosis and cell damage incurred during prion disease has been implied^{[26]}

A standard two-step immunohistochemical technique was established on tissue sections cut onto stainless steel tokens. A microglial, CD68 marker, monoclonal antibody, FA11 (Serotec), was implemented as the primary antibody and the fluorescent signal produced by the linkage of the biotinylated secondary antibody and an FITC—avidin complex, Neutravidin (Molecular Probes Inc., Eugene, Oreg., USA).

1.6 Prion Protein

Six monoclonal Scrapie associated fibril (SAF) antibodies (Table 1, FIG. 4) were provided by Prof J Grassi (CEA/Saclay).

The tissue was formalin-fixated and underwent two pre-treatments: Porous autoclaving for 20 minutes at 121° C., and formic acid (>95%) for 5 minutes, to destroy the normal form of the PrP protein (PrP^{c}) and reveal the epitopes of the aberrant PrP^{sc}. Table 1 SAF monoclonal antibodies and their epitopes on the Prp protein.

FIG. 4 Shows PrP Epitopes Recognised by the SAF Mab Antibodies in Relation to the Protein.

After pre-treatment the sections were processed according to the Vector Labs, M.O.M. kit procedure with a SAF incubation time of 2 h; an avidin—biotin complex was then applied and followed by the standard diaminobenzidine (DAB) reaction.

All of the initial characterisation work was performed on glass-mounted sections and all antibodies were tested on ME7, naïve, and NBH material. The SAF antibodies were then compared with the commercially available and well-characterised monoclonal antibody 6H4 (Prionics) antibody to assess their performance.

1.7 Direct Fluorochrome Staining.

To date no reagent has been applied to surface contamination or indeed detection of proteins on curved, ridged or smooth opaque or semi-opaque surfaces.

1.8 General Protein Contamination

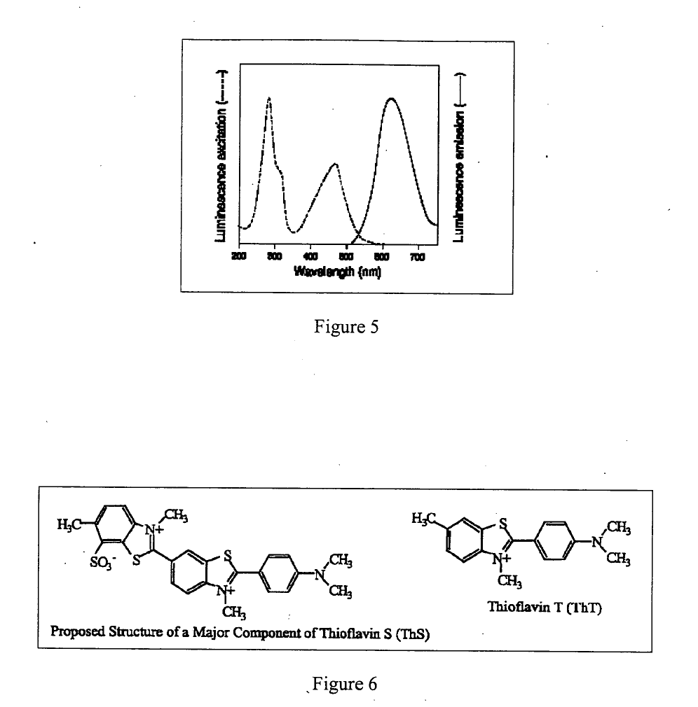

Spyro Ruby Fluorescent dye [Molecular Probes, Inc. Eugene, Oreg. 97402, USA] was developed for sensitive staining of low protein concentrations in gels only. General protein contamination was visualised by the modified application of Sypro Ruby stain. This stain has been show to possess a very high affinity for general proteins and be highly sensitive^{[27-32]} on both gels and glass surfaces. The excitation and emission spectra for Sypro Ruby are given below (FIG. 5).

Its successful application to tissue on metal tokens indicated its suitability as a hygiene screen for medical instruments. Therefore, subsequent implementation of the stain was initiated on discarded medical devices.

FIG. 5 Shows Sypro Ruby Excitation/Emission Spectra.

See also

Berggren, K. N et al ‘An improved formaultion of SYPRO Ruby protein gel stain: comparison with the original formulation and with a ruthenium II tris (Bathophenanthroline disulfonate) formulation’ Proteomics; 2 (5): 486-98, 2002.

Steinberg, T. H et al, ‘Rapid and simple single nanogram detection of glycoproteins in polyacrylamide gels and on electroblots’. Proteomics; I (7): 841-55, 2001.

Lopez, M. F et al. A comparison of silver stain and SYPRO Ruby Protein gel Stain with respect to protein detection in two-dimensional gels and identification by peptide mas profiling’. Electrophoresis; 21 917): 3673-83, 2000.

Berggren, K et al ‘A luminescent ruthenium complex for ultrasensitive detection of proteins immobilized on membrane support’ Anal. Biochem; 176(2): 129-43, 1999.

1.9 Prion Amyloid Protein

The thiazole derivatives, thioflavine T (ThT) and thioflavine S (ThS) (FIG. 6), have been shown to possess the ability to label amyloid deposits associated with amyloid plaques within histological sections from a number of neurodegenative diseases.^{[33-48]} Thiazoles's such as Thioflavine S and Thioflavine T have been used to detect amyloid deposits in fluids and post-mortem histological sections. Both ThT and ThS have emission spectra with maxima around 482 nm.^{[49]} ThS emission is stimulated by excitation at 385 nm, which is unchanged from that of the free dye in solution. However ThT, once bound, undergoes a change in excitation spectrum with a new peak appearing at 450 nm which does not exist for the free dye. This knowledge and the modification of the staining methodology to the application of the thiazole derivatives was investigated on 10 μm frozen sections placed onto glass slides and stainless steel tokens.

FIG. 6 Shows Thiazole Derivative Structures

1.10 Surface Contamination on ‘Clean’ Surgical Instruments.

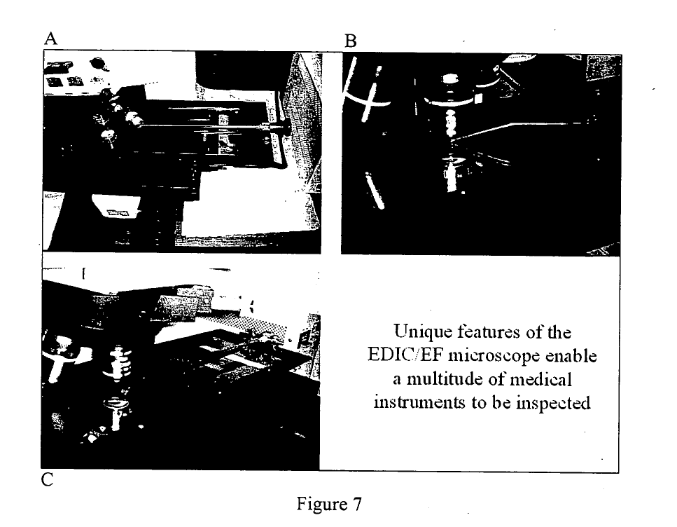

The versatile nature of the EDIC microscope enables a variety of different shaped and sized instruments to be scanned for contamination (FIG. 7).

The instruments used had passed through the regular cleaning, inspection and sterilisation procedures. Some instruments had been deemed unsatisfactory for theatre reuse. But none had been removed from circulation due to contamination.
A rigid cystoscope has a pencil thin extension and possesses both a light and lense at the tip to allow it to focus on the inner wall of the bladder or urethra. By this means the clinician is able to diagnose such conditions as: Persistant urinary tract infections
Haematuria
Incontinence
Interstitial cystitis, painful urination
Urethral blockage (Prostate enlargement), stricture or narrowing.

The cystoscope set was scanned using the EDIC microscope and then cleaned.

Results

1.11 Antibody Investigations

1.12 Microglia

Initial studies labelled ME7-infected neural tissue sections with a FITC-conjugated antibody raised against the CD68 marker to look for activated microglia.

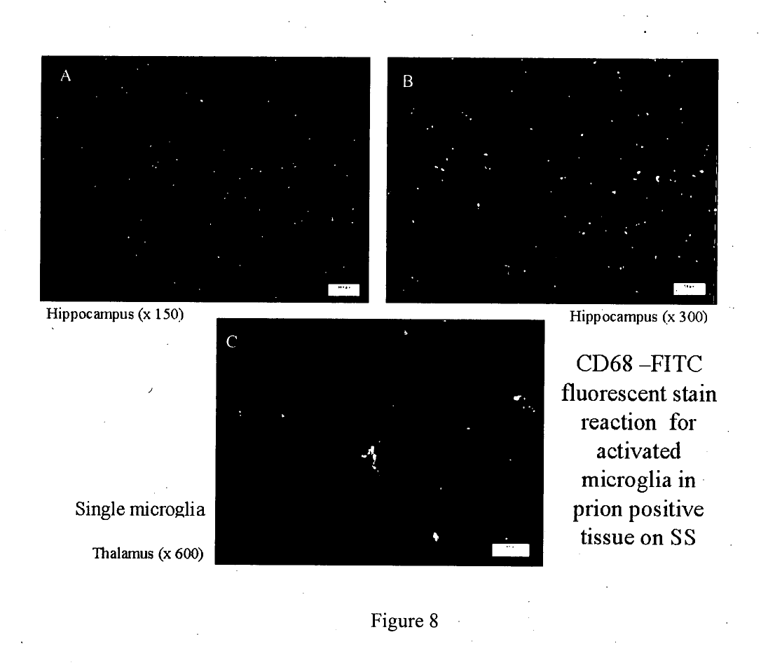

The microscopy revealed that extensive activation and recruitment of microglia occurred within sections of the hippocampus, and that these were clearly visible on brain sections mounted on stainless steel (FIGS. 8a and 8b). At greater magnification a single microglia and its processes can be visualised within the thalamus (FIG. 8c).

FIG. 8 Shows Activated Microglia on Stainless Steel Brain Sections.

1.13 Prion Protein

The dentate gyrus region of the hippocampus is a recognised area for PrP^{sc} deposition as can be seen from FIG. 9; the control tissue had no positive areas within the dentate gyrus (a). The application of SAF83 and SAF32 antibodies both gave a positive signal within the dentate gyrus (b), which is comparable to the findings obtained using the 6H4 Mab (c).

FIG. 9 Shows Positive SAF Areas in Dentate Gyrus Comparable to 6H4

Another known locus for PrP^{sc} aggregation in the ME7 model is the CA3 region of the hippocampus and this was also investigated (FIG. 10). Clearly the control tissue again has no positive areas within the CA3 region (a). The application of SAF83 and SAF32 antibodies both gave positive staining within the CA3 (c), which again are comparable to the findings from the 6H4 Mab (b).

FIG. 10 Shows Positive SAF Signal in the CA3 Region of the Hippocampus, Similar to 6H4.

A summary of the suitability of the six SAF antibodies and a comparison with 6H4 is given in Table 2. Two of the SAF antibodies appear most promising, SAF83 and SAF32. Both give good positive signal, although they appeared to produce greater background signal than the 6H4 monoclonal.

Conversely, it can be argued that this increase in signal may be caused by very small areas (sub-micron) of prion protein that the 6H4 does not detect; consequently the SAF antibodies are in essence more sensitive to such deposits.

Table 2 Shows the Suitability of the SAF Mab's for Staining PrP^{sc} in 10 μm Brain Sections. Direct Fluorochrome Staining

1.14 General Protein Contamination

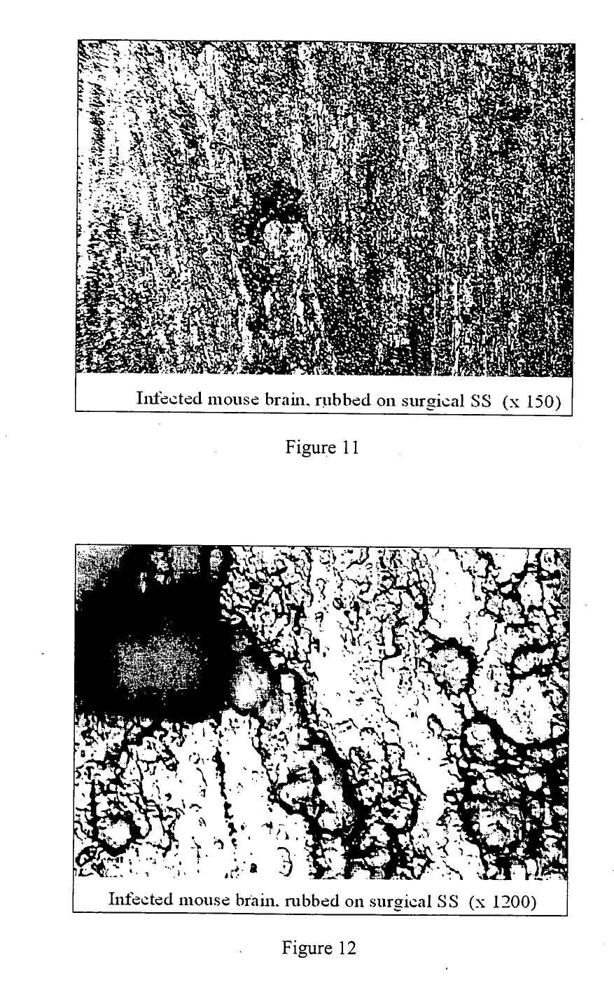

ME7-infected mouse brain was smeared on to a surgical stainless steel surface (FIG. 11) and visualised using EDIC microscopy at low power. The contrast between the neural deposition and the stainless steel can clearly be seen. The same brain smear at greater magnification is shown below (FIG. 12)

FIG. 11 Shows Low Magnification EDIC Image of Brain Contamination on Stainless Steel.

FIG. 12 Shows High Magnification EDIC Image of Brain Contamination on Stainless Steel.

1.15 Sypro Ruby Staining

FIG. 13 illustrates a brain-contaminated stainless steel token stained with a sensitive fluorescent protein marker (Sypro Ruby). The EDIC image of the brain contamination on the steel (a), the EF image after staining (b), and a software-derived composite picture of the two indicates the location of proteinaceous contamination.

FIG. 13 Shows Sypro Ruby Staining of Brain Contamination on Stainless Steel, Showing Protenaceous Material.

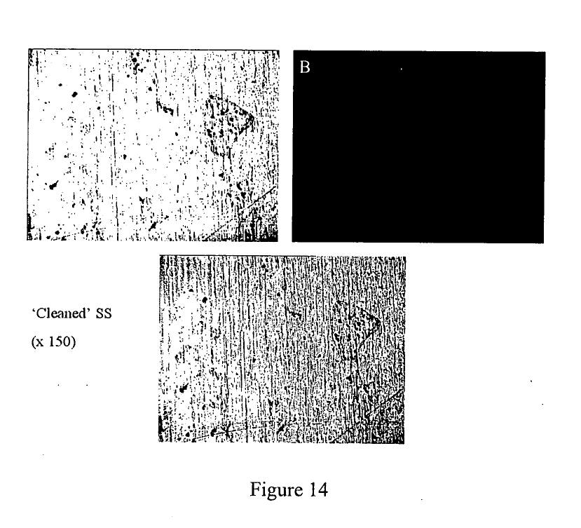

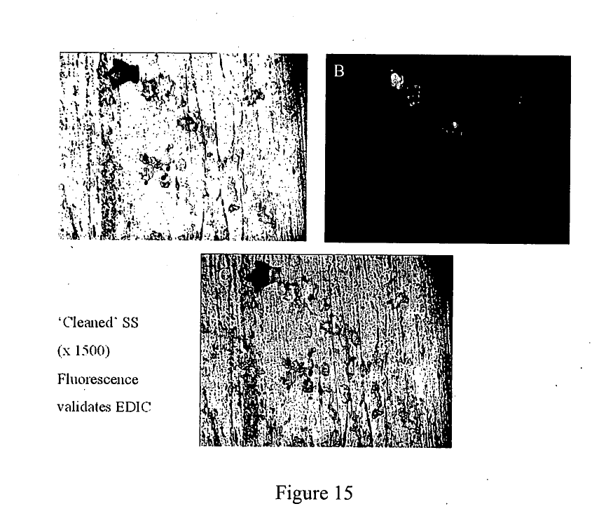

Surgical stainless steel surfaces were contaminated with brain material by smearing. These were then cleaned by the application of warm water, immersing in LabGuard scrub, rubbing with a surgical swab, and finally rinsing in warm water. The tokens were then stained with Sypro Ruby and examined under the microscope. At low magnification (FIG. 14), some contamination can be seen with just EDIC (a). Once stained with Sypro Ruby the contamination is made clearer (b) and when an analysis composite is produced the proteinaceous deposits size and location are clearly visible (c). FIG. 15 shows the same surface but under higher magnification. From this image a differentiation between lipid and protein contamination can be distinguished.

FIG. 14 Shows EDIC/EF Low Magnification Images Showing Detection, of Protein Deposits Remaining After Washing and Stained with Sypro Ruby.

FIG. 15 Shows High Magnification EDIC/EF Images Showing Detection of Protein Deposits Remaining After Washing and Staining with Sypro Ruby.

1.16 Prion Amyloid Protein

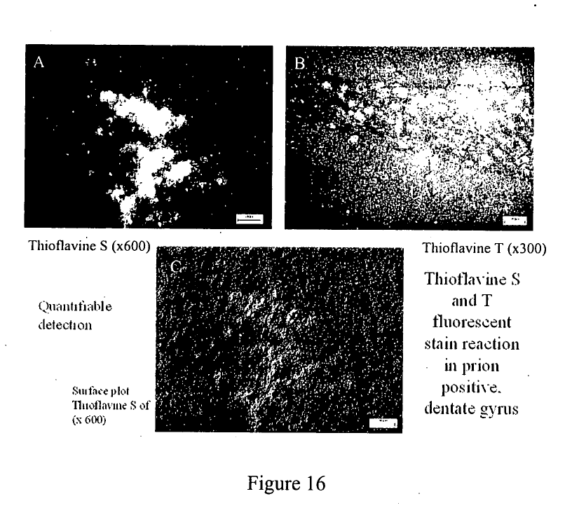

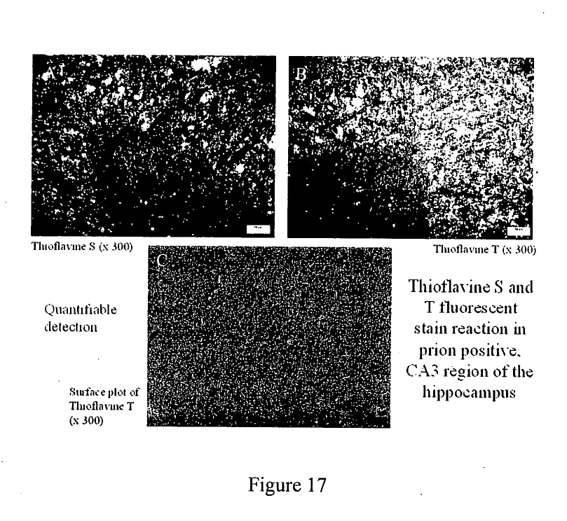

Representative PrP^{sc}-positive sections of brain were placed on surgical stainless steel surfaces and studied with thiazoles. A positive signal within the dentate gyrus (FIG. 16) and CA3 (FIG. 17) regions of the hippocampus can be seen. Thioflavine S (a) and Thioflavine T (b) staining are shown and a surface plot of the staining (c) can be produced to make the positive signal quantifiable. These positive areas are comparable to those seen within the immunohistological studies.

FIG. 16 Shows Thioflavine-Positive Regions in 10 μm Sections of Infected Dentate Gyrus on Stainless Steel.

FIG. 17 Shows Thioflavine-Positive Area in 10 μm Sections of Infected CA3 Region of Hippocampus on Stainless Steel.

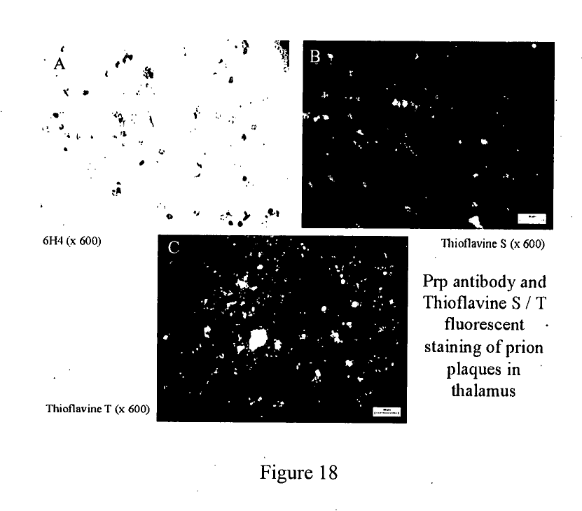

FIG. 18, Compares Directly the Immunohistological Staining by 6H4 Mab (a) and the Thioflavines (b,c) Within the Thalamus of Prion Positive Sections Placed Onto Surgical Stainless Steel Tokens.

FIG. 17 Shows Thioflavine-Positive Regions in 10 μm Sections of Infected Thalamus Comparable to 6H4 Mab Staining on Stainless Steel.

It can be seen from the previous figures that the minimum level of prion protein detection is less than 1 μm in diameter. This equates to less than 1 ρg (Appendix 1) of prion protein and demonstrates the sensitivity, ease and speed of the EDIC/EF staining techniques.

1.17 Surface Contamination on ‘Clean’ Surgical Instruments.

Spencer-Wells Forceps

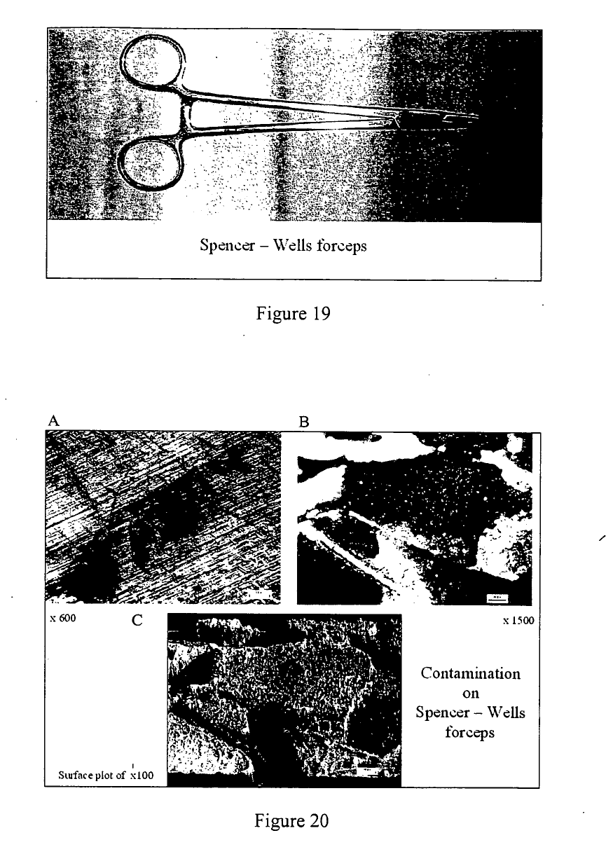

Spencer-Wells forceps (FIG. 19) are used extensively throughout the NHS, and applied for a variety of clamping procedures. EDIC microscopy displayed contamination on the outside of the forceps (FIG. 20). This visible contamination can be converted by image analysis into a surface plot to enhance the visualisation of the contamination.

FIG. 19 Shows Spencer Wells Forceps.

FIG. 20 Shows Contamination Found on Outside of the Spencer Wells Forceps.

1.18 Zoellner Sucker

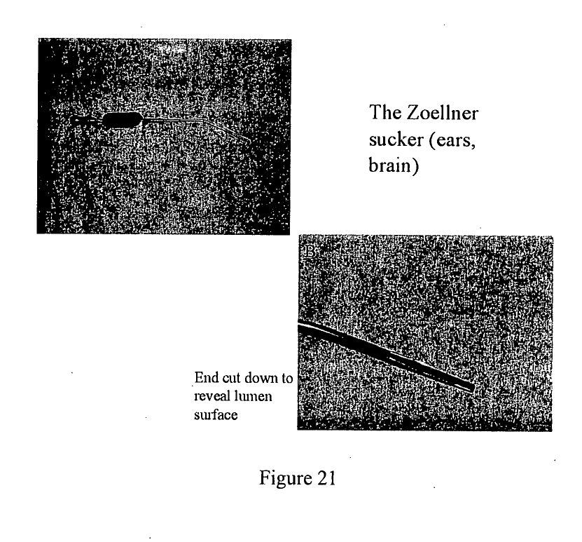

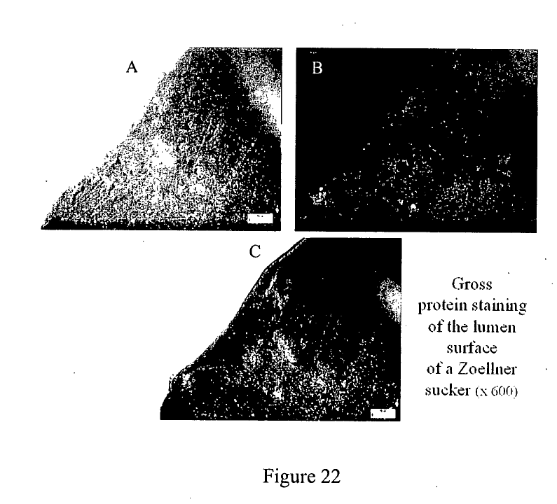

A Zoellner Sucker (FIGS. 21 and 22) is used for the removal of debris from ears and brain. The sucker had part of its outer casing cut down to reveal its lumenal surface. With solely EDIC microscopy (FIG. 22a), contamination is difficult to distinguish. However, after Sypro Ruby staining (FIG. 22b), and subsequent picture combination (FIG. 22c) the regions of proteinaceous deposition can be seen clearly.

FIG. 21 Shows Zoellner Sucker With Close Up Indicating Exposed Lumen.

FIG. 22 Shows Sypro Ruby Staining of Contamination on Lumenal Surface.

1.19 Cystoscope

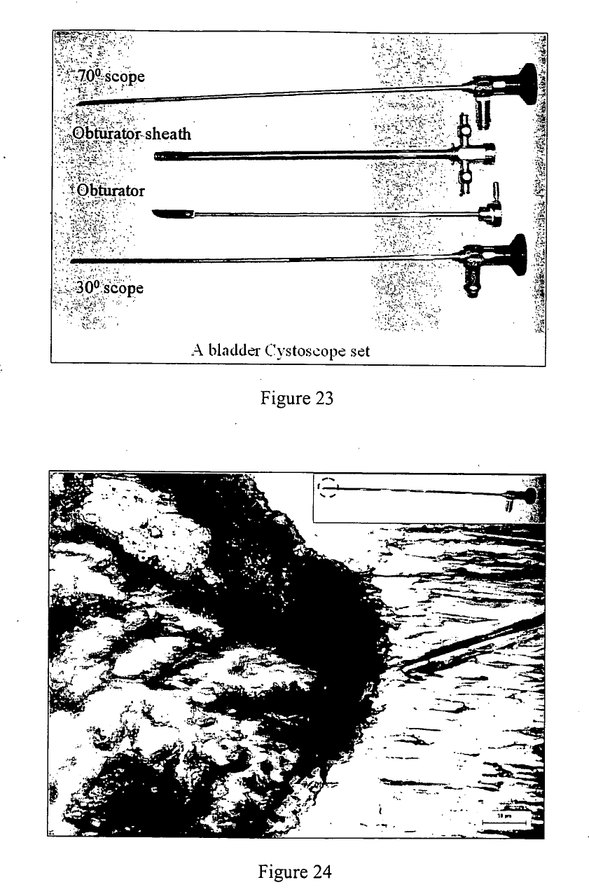

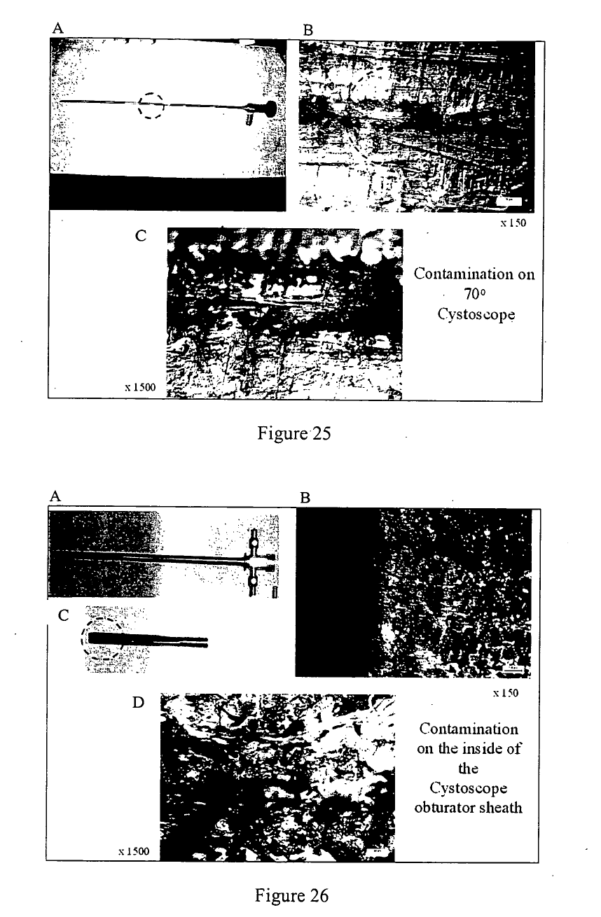

A bladder cystoscope set (FIG. 23) is comprised of four pieces: 2 scopes with different lens angulations, and an obturator and sheath to provide clear passage for the scopes into the area of interest. Initial EDIC investigation began on the scopes: visible contamination was discovered and an indication of this is readily demonstrated in FIGS. 24 and 25.

FIG. 23 Shows Cystoscope Set.

FIG. 24 Shows Contamination at Tip of 70° Cystoscope.

FIG. 25 Shows Contamination in the Middle of 70° Cystoscope.

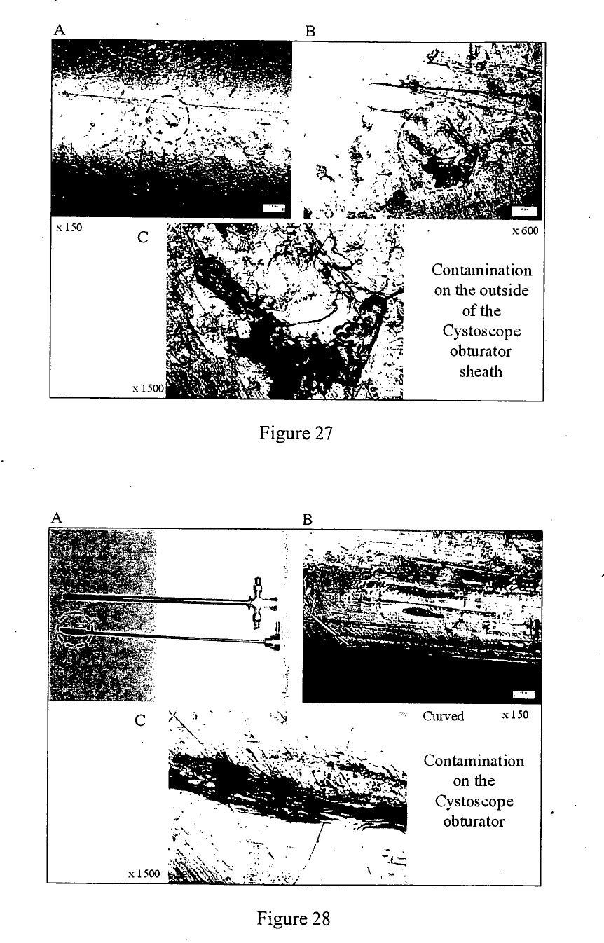

The obturator sheath was looked at both on the inside on the visible lumenal surface (FIG. 26) and the external surface (FIG. 27) The external scan picked up small areas of unknown deposits, whereas the extent of contamination on the lumen surface was such that ‘clean’ metal was difficult to visualise.

FIG. 26 Shows Contamination on Lumenal Surface of Cystoscope Set Obturator Sheath.

FIG. 27 Shows Contamination on Outside of Cystoscope Set Obturator Sheath.

The obturator tip was subsequently scanned, and small patches of contamination observed (FIG. 28). The obturator tip is highly curved and it is worth noting how readily this curvature can be visualised with the application of EDIC microscopy and software image analysis.

FIG. 28 Shows Contamination at Tip of Cystoscope Obturator.

### CONCLUSIONS

This study has provided evidence that EDIC/EF microscopy in conjunction with the appropriate probes can be used to reveal low levels of proteinaceous contamination on surgical stainless steel and on surgical instruments. The thiazoles, Thioflavine T and Thioflavine S, have been used to demonstrate contamination with β-pleated amyloid from prion diseased brain. Results have shown that the sensitivity of detection of β-amyloid on surgical stainless steel is comparable to that detected by immunocytochemical detection of PrP^{sc} with immuno-peroxidase methods.

The methods and techniques are:
Rapid and simple, being user friendly and enabling the scope for multi-environmental applications.
Sensitive technique able to detect sub-micron (<1 ρg) prion plaques
Quantitative and, inherently, able to provide a
Contamination Index for medical or industrial surfaces or tools.

The versatile nature of the EDIC/EF microscope and the extraordinary power of the microscope technique in conjunction with the reagents and antibodies, not only provides a sensitive scanning, and quality control device, but also provides as a valuable diagnostic aid.

### Example 2

### Screening Methods

The CMV antoigenaemia test is a rapid sensitive and quantifiable test useful in the early detection of CMV infections. Early detection allows clinicians to predict patients at risk and commence suitable treatment and monitoring.

The, T. H. van der Bij, W., van der Berg, A. P. van der Giessen, Weits, J., Sprenger, H. G. (1990). Cytomegalovirus Antigenaemia, Rev. Infect. Dis. 12 s 737 - 744

Gerna, G., Kiepto, D., Parea, M et al (1991). Cytomegalovirus infections and gangiclovir treatment in heart transplant recipients by determination of viraemia, antigenaemia and DNAemia, J. Infect. Dis. 164, 488-498.

The appearance of the Pk65 protein is one of the first signs of CMV disease. If it is possible to detect whether someone has CMV early enough then it is possible to give suitable drugs. A method whereby transplant patients (or other patient populations typically at risk) can be routinely screened through e.g., sampling of blood or other tissue, by looking for the variant protein can be a useful warning system.

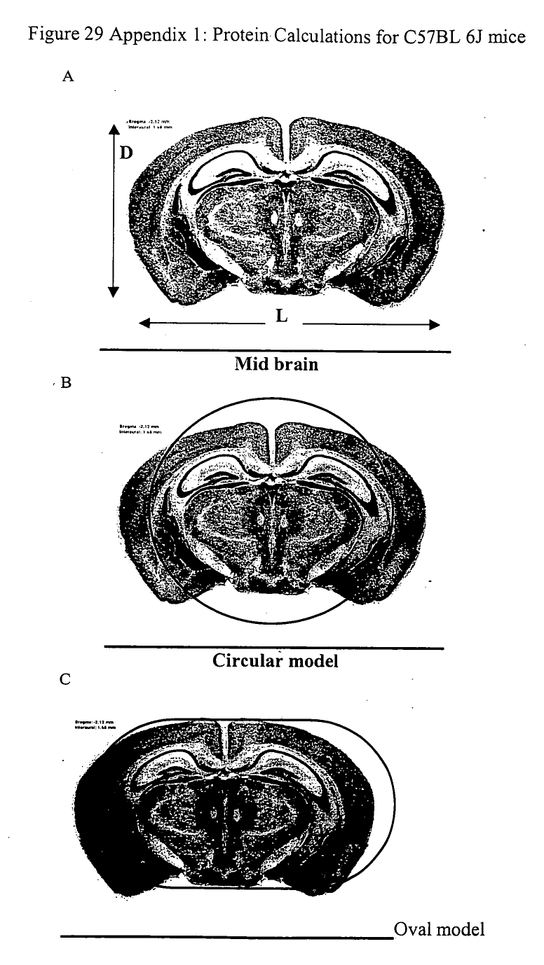

FIG. 29 Shows the View of Peripheral Blood Mononuclear Cells Stained with Monoclonal Antibody Against the Early Pk65 Protein of Cytomegalovirus (CMV).

MicroGen Bioproduct CMV antigenaemia kit (M83) Obtainable from Microgen Bioproducts Limited, Camberley, surrey.

### Example 3

### Microscope Configurations

The following figures further illustrate the versatility of the system. The microscope configurations referred to are those listed in Table 3.

Configuration 1

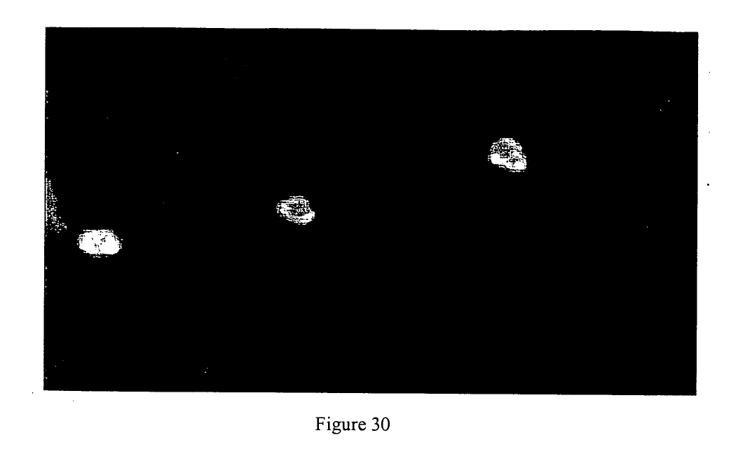

FIG. 30 CMV;

FIG. 31 CMV;

FIG. 32 Fluorescent image, high power (Hoffman);

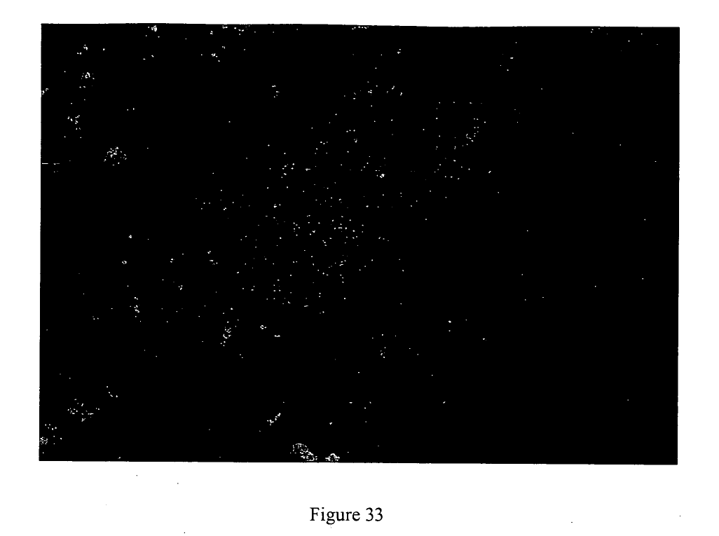

FIG. 33 Thioflavin T-stained dentate gyrus showing prion amyloid aggregates (FITC filter block) 1000× magnification;

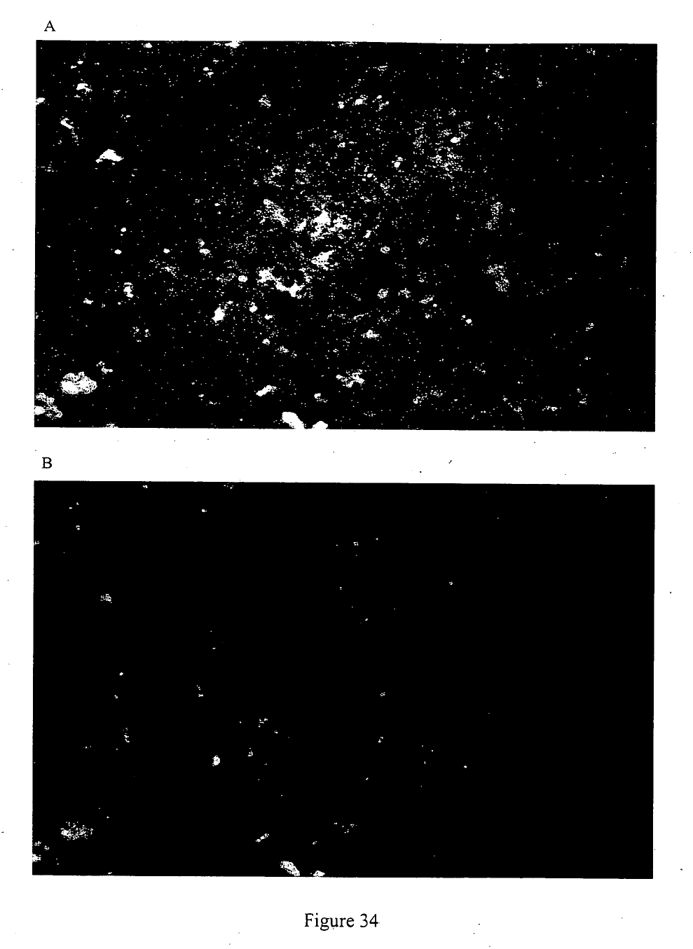

FIG. 34 Composite of thioflavin spyro ruby stained (TRITC filter block).

Configuration 3

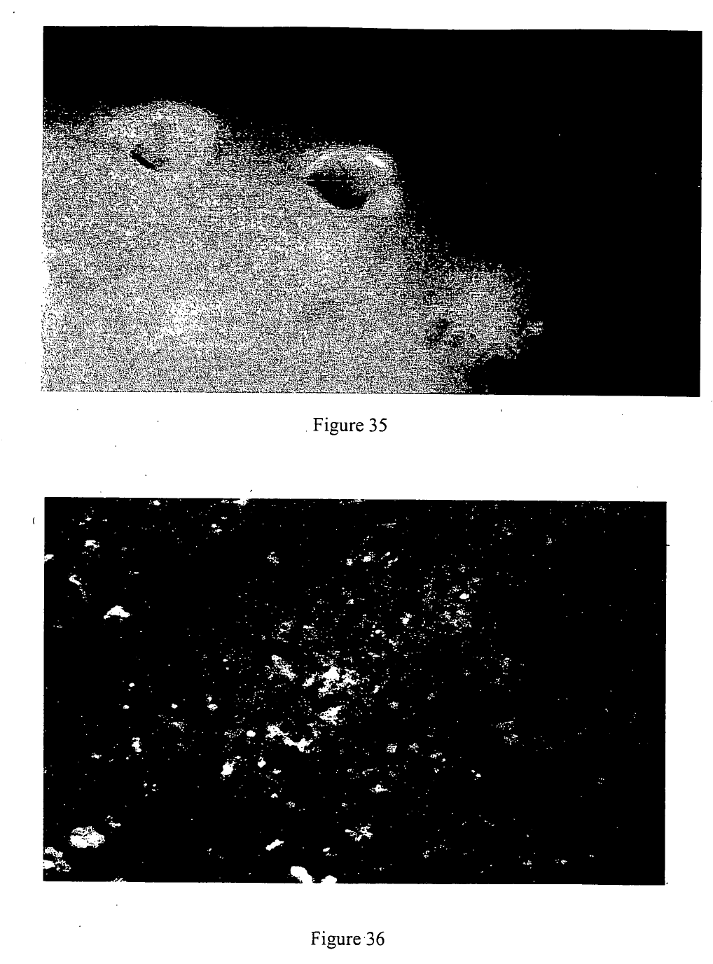

FIG. 35 Droplets excreted by fruit fly egg after laying, EDIC microscopy 1000× magnification.

Configuration 6/7

FIG. 36 Single focal plane EDIC/CONFOCAL microscopy (No 7 ) Optigrid

FIG. 37 Drinking water biofilm on polyethylene pipe, room temperature, composite of 10 stacked images. EDIC/Confocal microscopy (No. 7) Optigrid.

Configuration 8

FIG. 38 Spleen-Bright field 400× magnification

Configuration 9

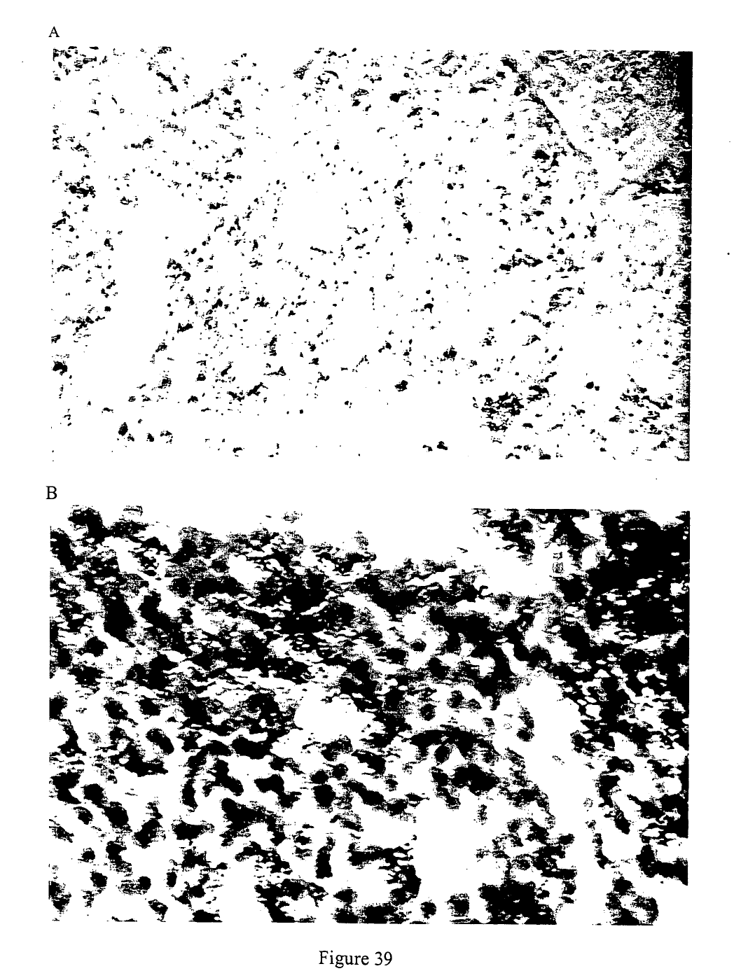

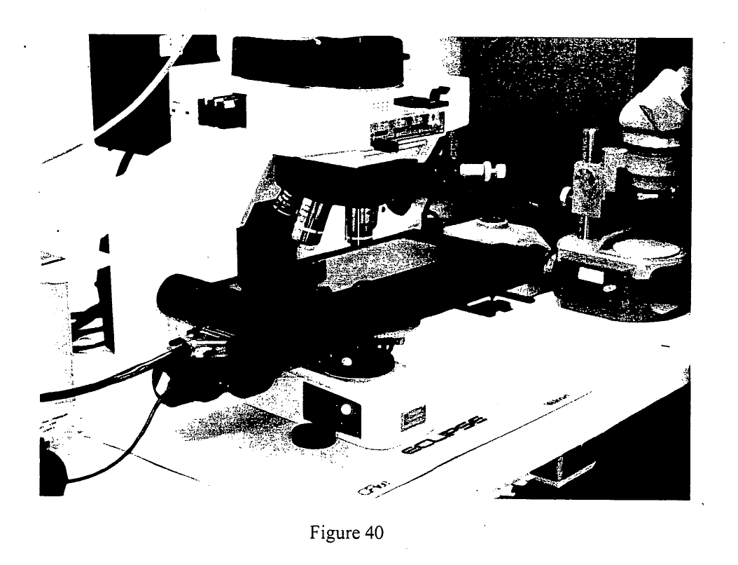

FIG. 39 Spleen-Hoffman modulation contrast, 1000× magnification

### REFERENCES

1. ACDP/SEAC, Transmissilbe Spongiform Encephalopathy Agents: Safe working and the prevention of infection. The stationary office, 1998.
2. Department of Health, U., Decontamination of medical devices. Health Services Circular, 2000. 2000/032.
3. Department of Health, U., Varient Creutzfeldt-Jakob disease (vCJD): Minimising the risk of transmission. Health Services Circular, 1999. 1999/178.
4. Department of Health, U., Controls assurance in infection control: Decontamination of medical devices. Health Services Circular, 1999. 1999/179.
5. Taylor, D. M., K. Fernie, and I. McConnell, Inactivation of the 22A strain of scrapie agent by autoclaving in sodium hydroxide. Vet Microbiol, 1997. 58(2-4): p. 87-91.
6. Taylor, D. M., et al., Decontamination studies with the agents of bovine spongiform encephalopathy and scrapie. Arch Virol, 1994. 139(3-4): p.313-26.
7. Zobeley, E., et al., Infectivity of scrapie prions bound to a stainless steel surface. Mol Med, 1999. 5(4): p. 240-3.
8. Bockman, J. M. and D. T. Kingsbury, Immunological analysis of host and agent effects on Creutzfeldt-Jakob disease and scrapie prion proteins. J Virol, 1988. 62(9): p. 3120-7.
9. Bruce, M. E., et al., The disease characteristics of different strains of scrapie in Sinc congenic mouse lines: implications for the nature of the agent and host control of pathogenesis. J Gen Virol, 1991. 72 (Pt 3): p. 595-603.
10. Bruce, M. E., et al., Strain characterization of natural sheep scrapie and comparison with BSE. J Gen Virol, 2002. 83(Pt 3): p. 695-704.
11. Carp, R. I., et al., Analysis of the incubation periods, induction of obesity and histopathological changes in senescence-prone and senescence-resistant mice infected with various scrapie strains. J Gen Virol, 1998. 79 (Pt 11): p. 2863-9.
12. Kim, Y. S., et al., Incubation periods and survival times for mice injected stereotaxically with three scrapie strains in different brain regions. J Gen Virol, 1987. 68 ( Pt 3): p. 695-702.
13. Kuczius, T., I. Haist, and M. H. Groschup, Molecular analysis of bovine spongiform encephalopathy and scrapie strain variation. J Infect Dis, 1998. 178(3): p. 693-9.
14. Kuczius, T. and M. H. Groschup, Differences in proteinase K resistance and neuronal deposition of abnormal prion proteins characterize bovine spongiform encephalopathy (BSE) and scrapie strains. Mol Med, 1999. 5(6): p. 406-18.
15. Somerville, R. A., et al., Characterization of thermodynamic diversity between transmissible spongiform encephalopathy agent strains and its theoretical implications. J Biol Chem, 2002. 277(13): p. 11084-9.
16. Betmouni, S., V. H. Perry, and J. L. Gordon, Evidence for an early inflammatory response in the central nervous system of mice with scrapie. Neuroscience, 1996. 74(1): p. 1-5.
17. Betmouni, S. and V. H. Perry, The acute inflammatory response in CNS following injection of prion brain homogenate or normal brain homogenate. Neuropathol Appl Neurobiol, 1999. 25(1): p. 20-8.
18. Brown, D. R., B. Schmidt, and H. A. Kretzschmar, Role of microglia and host prion protein in neurotoxicity of a prion protein fragment. Nature, 1996. 380(6572): p. 345-7.
19. Combrinck, M. I., V. H. Perry, and C. Cunningham, Peripheral infection evokes exaggerated sickness behaviour in pre-clinical murine prion disease. Neuroscience, 2002. 112(1): p. 7-11.
20. Heppner, F. L., M. Prinz, and A. Aguzzi, Pathogenesis of prion diseases: possible implications of microglial cells. Prog Brain Res, 2001. 132: p. 737-50.
21. Perry, V. H., et al., The blood-brain barrier and the inflammatory response. Mol Med Today, 1997. 3(8): p. 335-41.
22. Perry, V. H., C. Cunningham, and D. Boche, Atypical inflammation in the central nervous system in prion disease. Curr Opin Neurol, 2002. 15(3): p. 349-54.
23. Rezaie, P. and P. L. Lantos, Microglia and the pathogenesis of spongiform encephalopathies. Brain Res Brain Res Rev, 2001. 35(1): p. 55-72.
24. Russelakis-Carneiro, M., S. Betmouni, and V. H. Perry, Inflammatory response and retinal ganglion cell degeneration following intraocular injection of ME7. Neuropathol Appl Neurobiol, 1999. 25(3): p. 196-206.
25. Van Everbroeck, B., et al., The role of cytokines, astrocytes, microglia and apoptosis in Creutzfeldt-Jakob disease. Neurobiol Aging, 2002. 23(1): p. 59-64.
26. Giese, A., et al., Role of microglia in neuronal cell death in prion disease. Brain Pathol, 1998. 8(3): p. 449-57.
27. Berggren, K., et al., A luminescent ruthenium complex for ultrasensitive detection of proteins immobilized on membrane supports. Anal Biochem, 1999. 276(2): p. 129-43.
28. Berggren, K., et al., Background-free, high sensitivity staining of proteins in one- and two-dimensional sodium dodecyl sulfate-polyacrylamide gels using a luminescent ruthenium complex. Electrophoresis, 2000. 21(12): p. 2509-21.
29. Berggren, K. N., et al., An improved formulation of SYPRO Ruby protein gel stain:

Comparison with the original formulation and with a ruthenium II tris (bathophenanthroline disulfonate) formulation. Proteomics, 2002. 2(5): p. 486-98.
30. Kemper, C., et al., Simultaneous, two-color fluorescence detection of total protein profiles and beta-glucuronidase activity in polyacrylamide gel. Electrophoresis, 2001. 22(5): p. 970-6.
31. Steinberg, T. H., et al., Ultrasensitive fluorescence protein detection in isoelectric focusing gels using a ruthenium metal chelate stain. Electrophoresis, 2000. 21(3): p. 486-96.
32. Steinberg, T. H., et al., Rapid and simple single nanogram detection of glycoproteins in polyacrylamide gels and on electroblots. Proteomics, 2001. 1(7): p. 841-55.
33. Townsend, L., et al., Comparison of methods for analysis of CSF proteins in patients with Alzheimer's disease. Neurochem Pathol, 1987. 6(3): p. 213-29.
34. Klunk*, W. E., et al., Uncharged thioflavin-T derivatives bind to amyloid-beta protein with high affinity and readily enter the brain. Life Sci, 2001. 69(13): p. 1471-84.
35. Liberski, P. P., et al., Diagnosis of Alzheimer's disease with commercially available anti-beta peptide (beta A4) antibodies following microwave oven pretreatment. Pol J Pathol, 1995. 46(1): p. 43-6.
36. Mathis, C. A., et al., A lipophilic thioflavin-T derivative for positron emission tomography (PET) imaging of amyloid in brain. Bioorg Med Chem Lett, 2002. 12(3): p. 295-8.
37. McBride, P. A., M. E. Bruce, and H. Fraser, Immunostaining of scrapie cerebral amyloidplaques with antisera raised to scrapie-associated fibrils (SAF). Neuropathol Appl Neurobiol, 1988. 14(4): p. 325-36.
38. Naiki, H., et al., Fluorometric determination of amyloid fibrils in vitro using the fluorescent dye, thioflavin Tl. Anal Biochem, 1989. 177(2): p. 244-9.
39. Naiki, H., et al., Fluorometric examination of tissue amyloidfibrils in murine senile amyloidosis: use of the fluorescent indicator, thioflavine T. Lab Invest, 1990. 62(6): p. 768-73.
40. Piccardo*, P., et al., Proteinase-K-resistant prion protein isoforms in Gerstmann-Straussler-Scheinker disease (Indiana kindred). J Neuropathol Exp Neurol, 1996. 55(11): p. 1157-63.
41. Sun, A., X. V. Nguyen, and G. Bing, Comparative analysis of an improved thioflavin-s stain, Gallyas silver stain, and immunohistochemistry for neurofibrillary tangle demonstration on the same sections. J Histochem Cytochem, 2002. 50(4): p. 463-72.
42. Sun, A. N. X., Bing G, A novel fluorescent method for direct visualization of neurofibrillary pathology in Alzheimer's disease. J Neurosci Methods, 2001. 111(1): p. 17-27.
43. Saeed, S. and G. Fine, Thioflavin-T for amyloid detection. Am J Clin Path, 1967. 47(5): p. 588 -593.
44. Vallet, P. G., et al., A comparative study of histological and immunohistochemical methods for neurofibrillary tangles and senile plaques in Alzheimer's disease. Acta Neuropathol (Berl), 1992. 83(2): p. 170-8.
45. Vasser, P. and C. Culling, Fluorescent stains, with special reference to amyloid and connective tissue. A.M.A. Archives of Pathology, 1959. 68(23): p. 487-498.
46. Viriot, M. L. and J. C. Andre, Fluorescent dyes: a search for new tracers for hydrology. Analysis, 1989. 17(3): p. 97-111.
47. Wisniewski, H. M. and J. Wegiel, The neuropathology of Alzheimer's disease. Neuroimaging Clin N Am, 1995. 5(1): p. 45-57.
48. Hobbs, J. and A. Morgan, Fluorescence microscopy with thioflavine-T in the diagnosis of amyloid. J Path. Bact, 1963. 86: p. 437-442.
49. LeVine, H., 3rd, Thioflavine T interaction with synthetic Alzheimer's disease beta-amyloid peptides: detection of amyloid aggregation in solution. Protein Sci, 1993. 2(3): p. 404-10.
50. Shaked, G. M. et al, A protease-resistant prion protein isoform is present in urine of animals and humans affected with prion diseases. J Biol Chem, 2001. 276(34): p. 31479-31482.

The invention is further described by the following numbered paragraphs:
1. A microscopy method for use in the detection or identification of biological material on surfaces by implementing episcopic differential contrast (EDIC) microscopy plus epifluorescence (EF) microscopy wherein the microscope incorporates a DIC prism in the nosepiece, and an immuno-gold staining block and long distance objectives so that the materials can be visualised without requirement for a coverslip or oil or water immersion.
2. A method according to paragraph 1 wherein the surfaces are curved, ridged, smooth opaque or semi -opaque, transparent, fibrous, rough or corroded.
3. A method according to paragraph 1 or 2 wherein the surfaces are stainless steel instruments, surgical instruments, work surfaces, plastic surfaces, pipes or pipe biofilms, clothes, fabrics, food, grains, indwelling devices, biological samples, biopsy materials biofilms, membranes, interior of cells or exterior of cells.
4. A method according to paragraph 3 wherein the instruments are forceps, surgical knives or scalpels, rigid or flexible endoscopes, cytoscopes, applanation tonometer tips.
5. A method according to paragraph 3 wherein the indwelling devises are contact lenses, or catheters.
6. A method according to paragraph 1 wherein the biological material is protein contamination or biohazardous materials.
7. A method according to paragraph 1 wherein the biological materials are diseases.
8. A method according to paragraph 7 wherein the diseases are caused by bacteria or viruses.
9. A method according to paragraph 7 wherein the diseases are caused by amyloidogenic proteins.
10. A method according to paragraph 6 wherein the materials are helicobacter, campylobacteria, CMV, MRSA, TB, smallpox, or anthrax.
11. A method according to paragraph 6 wherein the diseases affect non-humans.
12. A method according to paragraph 11 wherein the diseases are BSE, Scrapie or deer/elk Chronic Wasting Disease.
13. A method according to any preceding paragraph wherein the biological materials bind fluorophores.
14. A method according to paragraph 6 wherein the viability/vitaility of the disease materials is determined using suitable staining techniques.
15. A method according to paragraph 13 wherein the staining technique is CTC or DAPI and/or PI.
16. A method according to paragraph 14 or 15 wherein the disease is cryptosporidium.
17. A method according to paragraph 13 wherein fluorescent reagents or specific biological probes are used.
18. A method according to paragraph 17 wherein the specific probes are monoclonal antibodies, peptides, nucleic acids or pseudonucleic acids.
19. A method according to paragraph 17 wherein the fluoorphores agents are fluorescent thiazole derivatives.
20. A method according to paragraph 19 wherein the derivatives are Thioflavine T or S.
21. A method according to paragraph 1 wherein general protein contamination is detected using Sypro Ruby fluorescent stain.
22. A method according to paragraph 8 wherein the detection level on stainless steel is less than 1 picogram of protein.
23. A microscope apparatus for use in the method according to any one of the preceding paragraphs.
24. A microscope according to paragraph 23 comprising an EDIC microscope with a high powered light system and a filter arranged in the light path so that light differences on the sample can be visualised.
25. A microscope according to paragraph 24 wherein the light system is a mercury lighting system.
26. A microscope according to paragraph 23, 24 or 25 including an immunogold staining block.
27. A Microscope apparatus for use in the method according to any one of paragraphs 1 to 22 comprising an EDIC microscope adapted to provide a handheld or portable device or for use in a conveyor belt or adapted for use in a modified containment cabinet.
28. A Microscope apparatus for use in the method according to any one of paragraphs 1 to 22 comprising an EDIC microscope which accommodates confocal adaptation without laser requirement.
29. A device adapted for screening in the water industry for the examination of biofilms, within medical establishments, contamination within the food industry, on food surfaces, abbatoirs, veterinary practices, dentistry practices comprising a microscope as defined in any one of paragraphs 23 to 28.
30. A kit for use in a method according to paragraph 1 for diagnostic screening for prion disease in patients after tissue biopsy.
31. A kit for use in a method according to paragraph 1 for quantitative assessment of the extent of contamination bound to surfaces comprising associated packs of reagents specifically designed to be used in conjunction with the method to enable visualisation of target cells.
32. A system for the diagnosis of disease including prion disease or any other amyloidogenic disease within bodily fluid of the human or animal subject, blood, urine, cerebral-spinal fluid, non-neuronal tissues (including spleen, lymph node), in cells, including living cells, using a method as defined in paragraph 1 or a microscope apparatus as defined in any of paragraphs 23 to 28.
33. A system for rapidly screening biofilms and assessing their contents using a method as defined in paragraph 1 or a microscope apparatus as defined in any of paragraphs 23 to 28.
34. A portable (handheld) or conveyor belt stage models to enable the rapid scanning of large surface areas or numerous articles in very short periods of time using a method as defined in paragraph 1 or a microscope apparatus as defined in any of paragraphs 23 to 28.
35. A quality control/safety scanner, able to rapidly visualise the structural integrity of opaque surfaces and tool/instruments there by quantifying the degree of pitting, scratching, etching or cracking that may have occurred, using a method as defined in paragraph 1 or a microscope apparatus as defined in any of paragraphs 23 to 28.
36. A method of assessing or validation of the effects or effectiveness of cleaning or disinfection methods on surfaces using a method as defined in paragraph 1 or a microscope apparatus as defined in any of paragraphs 23 to 28.
37. A kit for use in a method as defined in any of paragraphs 1, 32, or 36 comprising suitable probes for the biological material and/or any necessary stains.
   APPENDIX 1: Protein Calculations for C57BL 6J mice
   Models: Mid brain, Circular Model, Oval model (see FIG. 29)
   Normal Mouse Brain
   Theoretical

Brain Weight (whole brain): 482.3 mg (Av. gained from 129 mice within 23 littersⁱ)

Percentage of Protein in brain^{ii,iii,iv}: 12%

Brain Dimensions without olfactory lobe: 11 mm×5.5 mm (Dₐᵥ)×8 mm (Lₐᵥ)
(AP×DV×ML) (Averaged results (n=47)^{v})

Amount (g) of protein in average mouse brain=58 mg and Number of possible 10 μm sections˜1100 sections

Average protein/section=58/1100=52.6 μg/section

If we now assume each section is:a) circular
b) oval
the approx surface area of the sections can be calculated:

$$\begin{matrix}a ) Av . surface area ( circle ) = π {r}_{1}^{2} \\ ⇒ 3.142 \times {( {r}_{1} )}^{2} \\ ⇒ 3.142 \times {( ( ( {L}_{av} + {D}_{av} ) / 2 ) / 2 )}^{2} \\ ⇒ 3.142 \times {( 6.75 / 2 )}^{2} \\ ⇒ 3.142 \times {( 3.375 )}^{2} \\ = \underline{36 {mm}^{2}} \\ Av . protein per {mm}^{2} = 52.6 / 36 ⇒ \underline{1.46 μg \text{/} {mm}^{2}}\end{matrix}$$

$$\begin{matrix}b ) Av . surface area ( oval ) ⇒ ( area of av rectanglar middle + \\ area of Av circle ) \\ ⇒ ( {L}_{av} - {D}_{av} ) {D}_{av} + {π ( {D}_{av} / 2 )}^{2} \\ ⇒ ( 8 - 5.5 ) \times 5.5 + 23.8 \\ = \underline{37.5 {mm}^{2}}\end{matrix}$$

$$Av . protein per {mm}^{2} = 52.6 / 37.5 ⇒ \underline{1.40 μg \text{/} {mm}^{2}}$$

Software Derived Physical Measurement Comparison.

One section from the hippocampal area of the brain was calculated using both; Optilab, an area obtainment software package, and the mathematical modelling techniques described above.

The sections dimensions: 9 mm (L)×5.75 mm (D)

Optilab results indicated surface area of 48 mm²

$$\begin{matrix}a ) Circular = {( ( ( 9 + 5.75 ) / 2 ) / 2 )}^{2} \times π \\ = \underline{43 {mm}^{2}} \\ ⇒ \sim 10 \% difference \\ b ) Oval = ( 9 - 5.75 ) 5.75 + {π ( 5.75 / 2 )}^{2} \\ = ( 18.68 ) + ( 25.97 ) \\ = \underline{45 {mm}^{2}} \\ ⇒ \sim 6 \% difference\end{matrix}$$

This result would appear to indicate quite a close correlation between the mathematical and actual physical models for the brain.

As there is a greater correlation we will subsequently only consider the oval model.

Prion Mouse Brain

Theoretical

The theoretical models of the naive murine brain, were applied to prion disease brain. It was assumed that the outer dimensions of a prion brain are comparable to that of a naive brain, and that any weight loss occurred due to internal vascuolation.

Formalin fixed prion brains were weighed: Wtₚᵣ=463 mg (n=3)

NB: This value is approx 4% less than that of the naïve brain.

| | |
|---|---|
| Therefore 12% protein | [symbol] 55.6 mg protein |
| 1100 sections | [symbol] 50.5 μg protein/section |

Implementing the same surface area values gained from above using
only the oval model.

[symbol]

50.5/37.5 = 1.35 μg/mm² 1

```
Measurement of protein on the same plaque mentioned above
Protein in plaque = 20 × 10⁻⁶ mm² × 1.35 μg/mm² [symbol] ˜27 ρg
So for a 1 μm diameter plaque
Area of circle = π r² [symbol] π × (1 × 10⁻³/2)² mm² [symbol]
    ˜0.79 × 10⁻⁶ mm²
Protein in plaque = 0.79 × 10⁻⁶ mm² × 1.35 μg/mm² (oval) [symbol] ˜1 ρg
If we now attempt to equate this with actual prion molecules by
implementing Avrogadro's number (6 × 10²³), and
the molecular weight of the prion protein (approx. 30 kDA)
        1 Mol = 6 × 10²³ molecules = 30,000 g [symbol]
            (6 × 10²³/30000) molecules = 1 g [symbol]
            (2 × 10¹⁹/10¹²) molecules = 1 ρg [symbol]
            (2 × 10⁷ molecules = 1 ρg
```

Therefore as an infectious unit (IU) has been defined as 1×10⁵ prion molecules a 1 μm diameter plaque contains approx 200 infectious units.

Bio-Rad Protein Assay

Implementation of the Bio-Rad total protein assay was performed in order to assess the physical amount of protein within a section.

A 10 μm section was taken from the late (coronal) hippocampus region of an ME7-infected mouse. The sections dimensions were obtained from the stereotaxic atlas which indicated that the section possessed dimensions of approximately 9(L) mm×5.75 (D) mm.

The 10 μm section was homogenised in 50 μl of PBS and a 5 μl sample taken in accordance with the assay guidelines.

The Bio-Rad protocol was followed closely and an incubation time of 10 minutes was allowed.

The ELISA plate was then placed through the Dynex revelation 3.2 colourimeter (wavelength=630 nm) and the concentration of protein calculated in relation to the protein standards (standard curve value of 0.9928).

Results:

For a 10 μm section the system exported an average value of 1270 μg/ml

| | |
|---|---|
| in 5 μl | [symbol] (1270 × 5)/1000 (μg) |
| | [symbol] 6.4 μg |
| in 50 μl (i.e the section) | [symbol] 6.4 × 10 (μg/section) |
| | [symbol] 64 μg/section. 2 |

If we now calculate the surface area of the brain section and implement
our average protein/mm² values calculated above (1)

| | |
|---|---|
| Surface area of section | [symbol] ˜5.75 mm × 9 mm |
| | [symbol] 48 mm² (value from optilab) |

Protein content per section [symbol] 48 × 1.35 [symbol] 64.8 μg/section (oval model) 3
Clearly the calculated value 3 correlates well with the obtained value 2.
(˜1% difference)
Also as a further check that the value of 64 μg/section is a reasonable
value we can extrapolate this reading to cover the whole brain.

| | |
|---|---|
| 64 μg/section in 48 mm² section | [symbol] 1.33 μg/mm² |
| Therefore our av. section | [symbol] 1.33 × 37.5 |
| | [symbol] 49.9 μg/av. section |
| As the brain (1100 sections) | [symbol] 54.9 mg protein/brain |
| As protein = 12% | [symbol] 457 mg brain |
| | [symbol] ˜1% difference between this and the |
| | average prion brain weight. |

### References

¹Mouse Brain Library online.
¹Folch Pi, J., In ‘Biochemistry and the developing Nervous System, H Waelsch (ed), Academic press, New York; p 121 - 133, 1955.
¹Himwich W (Ed). ‘Biochemistry of the developing brain.’ Marcel Dekker Inc, New York; p 102 1973.
¹V O'Connor, Univ of Southampton (Verbal communication)

¹Franklin, K J. Paxinos, G. ‘The mouse brain in stereotaxic coordinates.’ Academic Press, London. 1997

TABLE 1
SAF monoclonal antibodies and their epitopes on the Prp protein

| Monoclonal | |
|---|---|
| Antibodies | Epitope |
| SAF 32 | 79-92 |
| SAF 34 | 79-92 |
| SAF 60 | 142-160 |
| SAF 61 | 142-160 |
| SAF 70 | 142-160 |
| SAF 83 | 126-164 |

TABLE 2
The suitability of the SAF Mab's for staining
PrP^{sc} in 10 μm brain sections

| Mab | Histochemical staining |
|---|---|
| 6H4 (Prionics) | ✓✓✓(✓) |
| SAF 32 (Grassi) | ✓✓✓ |
| SAF 34 | — |
| SAF 60 | ✓✓ |
| SAF 61 | ✓✓ |
| SAF 70 | — |
| SAF 83 | ✓✓✓ |

(✓) less background? Or SAF 32, 83 more sensitive for sub-micron plaques?

TABLE 3
EHU Microscope Configuration Definitions

| | No | Igs | Fluo | DIC | Con | Description | | | |
|---|---|---|---|---|---|---|---|---|---|
| Epi | 1 | x | √ | x | x | Epi - Fluorescence | | | |
| Illumination | 2 | √ | x | x | x | Epi - polarisation | | | |
| | 3 | √ | x | √ | x | EDIC | | | |
| | 4 | x | √ | √ | x | Epi - Fluorescence* | | | |
| | 5 | x | √ | x | √ | Epi - Fluorescent confocal | | | |
| | 6 | √ | x | x | √ | Epi - polarised confocal | | | |
| | 7 | √ | x | √ | √ | EDIC confocal | | | |
| | | | | | | Hoffman | | B.F | |
| | | Igs | Fluo | DIC | Con | Cond | Obj | Cond | |
| Trans − | 8 | x | x | x | x | x | x | √ | Brightfield |
| illumination | 9 | x | x | x | x | √ | √ | x | Hoffman |
| Trans + Epi | 10 | √ | x | x | x | √ | √ | x | Epi-polarised/Hoffman |
| illumination | 11 | x | √ | x | x | √ | √ | x | Epi-fluor/Hoffman |
| | 12 | √ | x | √ | x | √ | √ | x | Hoffman/EDIC |
| | 13 | √ | x | x | √ | √ | √ | x | Brightfield confocal/ |
| | | | | | | | | | Hoffman |
| | 14 | √ | x | x | √ | x | x | √ | Brightfield confocal |
| | 15 | √ | x | x | x | √ | x | x | Epi-polarised/pseudo |
| | | | | | | | | | Hoffman |
| | 16 | x | √ | x | x | √ | x | x | Epi-fluor/pseudo |
| | | | | | | | | | Hoffman |
| | 17 | √ | x | √ | x | √ | x | x | Pseudo Hoffman/EDIC |
| | 18 | √ | x | x | √ | √ | x | x | Brightfield confocal/ |
| | | | | | | | | | pseudo Hoffman |

Inferior to No. 1 therefore not applied
Table 3 Key:
Igs—Immuno-gold staining filter block,
DIC—Normarski Prism
B.F.—Brightfield
bj—Objective
Fluo—Fluorescent filter block
Con—Confocal (Thales - Optem)
Cond—Condenser

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A method for detecting biological material on a surface comprising using episcopic differential contrast (EDIC) microscopy plus epifluorescence (EF) microscopy, wherein the microscope comprises a DIC prism in the nosepiece, and an immuno-gold staining block and long distance objectives, wherein the biological material can be visualised without a coverslip and without oil or water immersion of the objectives.

2. The method according to claim 1, wherein the surface is curved, ridged, smooth, opaque, semi-opaque, transparent, fibrous, rough or corroded.

3. The method according to claim 1, wherein the surface is of an object selected from the group consisting of stainless steel instruments, surgical instruments, work surfaces, plastic surfaces, pipes, pipe biofilms, clothes, fabrics, food, grains, indwelling devices, biological samples, biopsy materials biofilms, membranes, interior of cells and exterior of cells.

4. The method according to claim 3, wherein the stainless steel instruments or surgical instruments are selected from the group consisting of forceps, surgical knives, scalpels, rigid endoscopes, flexible endoscopes, cytoscopes, and applanation tonometer tips.

5. The method according to claim 3, wherein less than 1 picogram of protein is detected on stainless steel instruments.

6. The method according to claim 3, wherein the indwelling devices are contact lenses or catheters.

7. The method according to claim 1, wherein the biological material is a protein, a pathogen or cells infected therewith.

8. The method according to claim 7, wherein the protein is amyloidogenic protein.

9. The method according to claim 7, wherein the pathogen is selected from the group consisting of helicobacter, campylobacteria, cytomegalogvirus (CMV), methicillin resistant Staphylococcus aureus (MRSA), Mycobacterium tuberculosis (TB), smallpox, anthrax, Cryptosporidium, bovine spongiform encephalopathy (BSE), Scrapie and deer/elk Chronic Wasting Disease.

10. The method according to claim 1, wherein the biological material binds a fluorophore.

11. The method according to claim 10, wherein the fluorophore is selected from the group consisting of 5-cyano-2,3-ditolyl-tetrazolium chloride (CTC), 4′,6′-diamidino-2-phenylindole hydrochloride (DAPI), propidium iodide (PI), Spyro Ruby and fluorescent thiazole derivatives.

12. The method according to claim 10, wherein viability or vitality of the biological material is determined using CTC, DAPI and/or PI.

13. The method according to claim 1, wherein the biological material is detected using monoclonal antibodies, peptides, nucleic acids or pseudonucleic acids.

14. The method according to claim 1, wherein detecting the biological material diagnoses prion disease or other amyloidogenic disease within bodily fluid, non-neuronal tissue or cells of a human or animal subject.

15. The method according to claim 1, wherein detecting the biological material assesses effectiveness of cleaning or disinfection methods on the surfaces.

16. A microscope comprising an EDIC microscope with a high-powered light system and a filter arranged in the light path so that light differences on the surface of a sample can be visualised.

17. The microscope according to claim 16, wherein the light system is a mercury light system.

18. The microscope according to claim 16, further comprising an immuno-gold staining block.

19. The microscope according to claim 16, wherein the microscope is adapted to provide a handheld or portable device or for use in a conveyor belt or is adapted for use in a modified containment cabinet.

20. The microscope according to claim 16, wherein the microscope is adapted for confocal microscopy without requiring a laser.
